SEQUENCE LISTING (337 sequences)

SEQ ID NO: 1 - DNA, 11 bp, Artificial Sequence
  Description of Artificial Sequence Synthetic oligonucleotide
tgttattcagt

SEQ ID NO: 2 - DNA, 12 bp, Artificial Sequence
  Description of Artificial Sequence Synthetic oligonucleotide
aacgtgactttt

SEQ ID NO: 3 - DNA, 9 bp, Artificial Sequence
  Description of Artificial Sequence Synthetic oligonucleotide
attgtatct

SEQ ID NO: 4 - DNA, 17 bp, Artificial Sequence
  Description of Artificial Sequence Synthetic oligonucleotide
gtcctttattgcaagaa

SEQ ID NO: 5 - DNA, 10 bp, Artificial Sequence
  Description of Artificial Sequence Synthetic oligonucleotide
actggaattc

SEQ ID NO: 6 - DNA, 14 bp, Artificial Sequence
  Description of Artificial Sequence Synthetic oligonucleotide
ttagttcatagcat

SEQ ID NO: 7 - DNA, 13 bp, Artificial Sequence
  Description of Artificial Sequence Synthetic oligonucleotide
atgtattattccg

SEQ ID NO: 8 - DNA, 10 bp, Artificial Sequence
  Description of Artificial Sequence Synthetic oligonucleotide
attatggcac

SEQ ID NO: 9 - DNA, 8 bp, Artificial Sequence
  Description of Artificial Sequence Synthetic oligonucleotide
catagttt

SEQ ID NO: 10 - DNA, 14 bp, Artificial Sequence
  Description of Artificial Sequence Synthetic oligonucleotide
atttcctgtaatag

SEQ ID NO: 11 - DNA, 12 bp, Artificial Sequence
  Description of Artificial Sequence Synthetic oligonucleotide
gatgtctcatta

SEQ ID NO: 12 - DNA, 14 bp, Artificial Sequence
  Description of Artificial Sequence Synthetic oligonucleotide
ctacgtttttagaa

SEQ ID NO: 13 - DNA, 12 bp, Artificial Sequence
  Description of Artificial Sequence Synthetic oligonucleotide
taagcgtttcat

SEQ ID NO: 14 - DNA, 14 bp, Artificial Sequence
  Description of Artificial Sequence Synthetic oligonucleotide
gaccgtattaattt

SEQ ID NO: 15 - DNA, 13 bp, Artificial Sequence
  Description of Artificial Sequence Synthetic oligonucleotide
attagtttctgac

SEQ ID NO: 16 - DNA, 18 bp, Artificial Sequence
  Description of Artificial Sequence Synthetic oligonucleotide
gtatccattatgagtatc

SEQ ID NO: 17 - DNA, 8 bp, Artificial Sequence
  Description of Artificial Sequence Synthetic oligonucleotide
ttttcgaa

SEQ ID NO: 18 - DNA, 16 bp, Artificial Sequence
  Description of Artificial Sequence Synthetic oligonucleotide
ttctacgatgctaatg

SEQ ID NO: 19 - DNA, 13 bp, Artificial Sequence
  Description of Artificial Sequence Synthetic oligonucleotide
catgatattctgt

SEQ ID NO: 20 - DNA, 19 bp, Artificial Sequence
  Description of Artificial Sequence Synthetic oligonucleotide
attaatgtccaagtttgct

SEQ ID NO: 21 - DNA, 17 bp, Artificial Sequence
  Description of Artificial Sequence Synthetic oligonucleotide
ttgcattagttcaaacg

SEQ ID NO: 22 - DNA, 9 bp, Artificial Sequence
  Description of Artificial Sequence Synthetic oligonucleotide
tacttttag

SEQ ID NO: 23 - DNA, 16 bp, Artificial Sequence
  Description of Artificial Sequence Synthetic oligonucleotide
gttgctaaagcattct

SEQ ID NO: 24 - DNA, 16 bp, Artificial Sequence
  Description of Artificial Sequence Synthetic oligonucleotide
gagctatgtttcaatc

SEQ ID NO: 25 - DNA, 10 bp, Artificial Sequence
  Description of Artificial Sequence Synthetic oligonucleotide
atccgtagta

SEQ ID NO: 26 - DNA, 11 bp, Artificial Sequence
  Description of Artificial Sequence Synthetic oligonucleotide
attgacgtcat

SEQ ID NO: 27 - DNA, 12 bp, Artificial Sequence
  Description of Artificial Sequence Synthetic oligonucleotide
gctgttcaatat

SEQ ID NO: 28 - DNA, 14 bp, Artificial Sequence
  Description of Artificial Sequence Synthetic oligonucleotide
tacggttacattta

SEQ ID NO: 29 - DNA, 15 bp, Artificial Sequence
  Description of Artificial Sequence Synthetic oligonucleotide
tattacgctagagct

SEQ ID NO: 30 - DNA, 15 bp, Artificial Sequence
  Description of Artificial Sequence Synthetic oligonucleotide
gtatatccgctgtaa

SEQ ID NO: 31 - DNA, 17 bp, Artificial Sequence
  Description of Artificial Sequence Synthetic oligonucleotide
cgtctaattaggacatt

SEQ ID NO: 32 - DNA, 20 bp, Artificial Sequence
  Description of Artificial Sequence Synthetic oligonucleotide
tcatatattactaggacggc

SEQ ID NO: 33 - DNA, 9 bp, Artificial Sequence
  Description of Artificial Sequence Synthetic oligonucleotide
tatactttg

SEQ ID NO: 34 - DNA, 15 bp, Artificial Sequence
  Description of Artificial Sequence Synthetic oligonucleotide
tatgtgactcaatcg

SEQ ID NO: 35 - DNA, 20 bp, Artificial Sequence
  Description of Artificial Sequence Synthetic oligonucleotide
atcaagcttcgtagtgctaa

SEQ ID NO: 36 - DNA, 15 bp, Artificial Sequence
  Description of Artificial Sequence Synthetic oligonucleotide
tacattagccgtagt

SEQ ID NO: 37 - DNA, 11 bp, Artificial Sequence
  Description of Artificial Sequence Synthetic oligonucleotide
tatccagtagt

SEQ ID NO: 38 - DNA, 11 bp, Artificial Sequence
  Description of Artificial Sequence Synthetic oligonucleotide
ttatggcctaa

SEQ ID NO: 39 - DNA, 9 bp, Artificial Sequence
  Description of Artificial Sequence Synthetic oligonucleotide
gattcttta

SEQ ID NO: 40 - DNA, 16 bp, Artificial Sequence
  Description of Artificial Sequence Synthetic oligonucleotide
aatcgtatccagtgtt

SEQ ID NO: 41 - DNA, 20 bp, Artificial Sequence
  Description of Artificial Sequence Synthetic oligonucleotide
tggtaaaactagactccgtt

SEQ ID NO: 42 - DNA, 16 bp, Artificial Sequence
  Description of Artificial Sequence Synthetic oligonucleotide
ttagcgtattatcagc

SEQ ID NO: 43 - DNA, 20 bp, Artificial Sequence
  Description of Artificial Sequence Synthetic oligonucleotide
gttgactaatgagcactatc

SEQ ID NO: 44 - DNA, 10 bp, Artificial Sequence
  Description of Artificial Sequence Synthetic oligonucleotide
cttagatagc

SEQ ID NO: 45 - DNA, 14 bp, Artificial Sequence
  Description of Artificial Sequence Synthetic oligonucleotide
aattacagtttctg

SEQ ID NO: 46 - DNA, 11 bp, Artificial Sequence
  Description of Artificial Sequence Synthetic oligonucleotide
agcatatttgc

SEQ ID NO: 47 - DNA, 14 bp, Artificial Sequence
  Description of Artificial Sequence Synthetic oligonucleotide
atgtcactatgtta

SEQ ID NO: 48 - DNA, 13 bp, Artificial Sequence
  Description of Artificial Sequence Synthetic oligonucleotide
tcacttatgtgta

SEQ ID NO: 49 - DNA, 18 bp, Artificial Sequence
  Description of Artificial Sequence Synthetic oligonucleotide
ccaagtttttcagagtta

SEQ ID NO: 50 - DNA, 9 bp, Artificial Sequence
  Description of Artificial Sequence Synthetic oligonucleotide
atcagtttt

SEQ ID NO: 51 - DNA, 18 bp, Artificial Sequence
  Description of Artificial Sequence Synthetic oligonucleotide
tgctttaatatccaggat

SEQ ID NO: 52 - DNA, 19 bp, Artificial Sequence
  Description of Artificial Sequence Synthetic oligonucleotide
tttttgacaataatgcgtc

SEQ ID NO: 53 - DNA, 12 bp, Artificial Sequence
  Description of Artificial Sequence Synthetic oligonucleotide
taatatgcttcg

SEQ ID NO: 54 - DNA, 18 bp, Artificial Sequence
  Description of Artificial Sequence Synthetic oligonucleotide
aatatgtaccgttgtcat

SEQ ID NO: 55 - DNA, 9 bp, Artificial Sequence
  Description of Artificial Sequence Synthetic oligonucleotide
tgtattcta

SEQ ID NO: 56 - DNA, 14 bp, Artificial Sequence
  Description of Artificial Sequence Synthetic oligonucleotide
acatacttaggttt

SEQ ID NO: 57 - DNA, 19 bp, Artificial Sequence
  Description of Artificial Sequence Synthetic oligonucleotide
tagccttcagtaagtttat

SEQ ID NO: 58 - DNA, 16 bp, Artificial Sequence
  Description of Artificial Sequence Synthetic oligonucleotide
agatctagtgcttcat

SEQ ID NO: 59 - DNA, 12 bp, Artificial Sequence
  Description of Artificial Sequence Synthetic oligonucleotide
atttgccttgaa

SEQ ID NO: 60 - DNA, 13 bp, Artificial Sequence
  Description of Artificial Sequence Synthetic oligonucleotide
tgtctgactttaa

SEQ ID NO: 61 - DNA, 10 bp, Artificial Sequence
  Description of Artificial Sequence Synthetic oligonucleotide
gatatacctg

SEQ ID NO: 62 - DNA, 18 bp, Artificial Sequence
  Description of Artificial Sequence Synthetic oligonucleotide
actgaatgattattcctg

SEQ ID NO: 63 - DNA, 19 bp, Artificial Sequence
  Description of Artificial Sequence Synthetic oligonucleotide
gtcggtatttaatcactat

SEQ ID NO: 64 - DNA, 16 bp, Artificial Sequence
  Description of Artificial Sequence Synthetic oligonucleotide
attcgatcgtcatgat

SEQ ID NO: 65 - DNA, 9 bp, Artificial Sequence
  Description of Artificial Sequence Synthetic oligonucleotide
ttcgtaatt

SEQ ID NO: 66 - DNA, 13 bp, Artificial Sequence
  Description of Artificial Sequence Synthetic oligonucleotide
tcgtttaatgcat

SEQ ID NO: 67 - DNA, 12 bp, Artificial Sequence
  Description of Artificial Sequence Synthetic oligonucleotide
tcagatcttatg

SEQ ID NO: 68 - DNA, 19 bp, Artificial Sequence
  Description of Artificial Sequence Synthetic oligonucleotide
agttcttaaccgtatattg

SEQ ID NO: 69 - DNA, 15 bp, Artificial Sequence
  Description of Artificial Sequence Synthetic oligonucleotide
aatctcctaattggg

SEQ ID NO: 70 - DNA, 14 bp, Artificial Sequence
  Description of Artificial Sequence Synthetic oligonucleotide
tctaaggcatttat

SEQ ID NO: 71 - DNA, 13 bp, Artificial Sequence
  Description of Artificial Sequence Synthetic oligonucleotide
atctttagtgact

SEQ ID NO: 72 - DNA, 11 bp, Artificial Sequence
  Description of Artificial Sequence Synthetic oligonucleotide
aattgcgatct

SEQ ID NO: 73 - DNA, 12 bp, Artificial Sequence
  Description of Artificial Sequence Synthetic oligonucleotide
cgatcagtttat

SEQ ID NO: 74 - DNA, 20 bp, Artificial Sequence
  Description of Artificial Sequence Synthetic oligonucleotide
gcggtcaatatgctacatta

SEQ ID NO: 75 - DNA, 15 bp, Artificial Sequence
  Description of Artificial Sequence Synthetic oligonucleotide
cgctatttagaatgc

SEQ ID NO: 76 - DNA, 16 bp, Artificial Sequence
  Description of Artificial Sequence Synthetic oligonucleotide
gtctgatatacagtct

SEQ ID NO: 77 - DNA, 9 bp, Artificial Sequence
  Description of Artificial Sequence Synthetic oligonucleotide
ttacgattt

SEQ ID NO: 78 - DNA, 20 bp, Artificial Sequence
  Description of Artificial Sequence Synthetic oligonucleotide
agagtatcgtcgaattacct

SEQ ID NO: 79 - DNA, 8 bp, Artificial Sequence
  Description of Artificial Sequence Synthetic oligonucleotide
catgttat

SEQ ID NO: 80 - DNA, 12 bp, Artificial Sequence
  Description of Artificial Sequence Synthetic oligonucleotide
acgtatttactg

SEQ ID NO: 81 - DNA, 15 bp, Artificial Sequence
  Description of Artificial Sequence Synthetic oligonucleotide
ttcgtacctaagtag

SEQ ID NO: 82 - DNA, 12 bp, Artificial Sequence
  Description of Artificial Sequence Synthetic oligonucleotide
cagtgcttaatt

SEQ ID NO: 83 - DNA, 20 bp, Artificial Sequence
  Description of Artificial Sequence Synthetic oligonucleotide
cttcacaattgtactgggaa

SEQ ID NO: 84 - DNA, 16 bp, Artificial Sequence
  Description of Artificial Sequence Synthetic oligonucleotide
tattactgggcactat

SEQ ID NO: 85 - DNA, 20 bp, Artificial Sequence
  Description of Artificial Sequence Synthetic oligonucleotide
acatcttcggcaatttgaag

SEQ ID NO: 86 - DNA, 19 bp, Artificial Sequence
  Description of Artificial Sequence Synthetic oligonucleotide
agatttccagtctgtatta

SEQ ID NO: 87 - DNA, 16 bp, Artificial Sequence
  Description of Artificial Sequence Synthetic oligonucleotide
gttacctaatgctagt

SEQ ID NO: 88 - DNA, 12 bp, Artificial Sequence
  Description of Artificial Sequence Synthetic oligonucleotide
catttaatggtc

SEQ ID NO: 89 - DNA, 8 bp, Artificial Sequence
  Description of Artificial Sequence Synthetic oligonucleotide
tactttag

SEQ ID NO: 90 - DNA, 10 bp, Artificial Sequence
  Description of Artificial Sequence Synthetic oligonucleotide
cgattcaagt

SEQ ID NO: 91 - DNA, 10 bp, Artificial Sequence
  Description of Artificial Sequence Synthetic oligonucleotide
tatacagtgc

SEQ ID NO: 92 - DNA, 15 bp, Artificial Sequence
  Description of Artificial Sequence Synthetic oligonucleotide
tccaaggttaatgtc

SEQ ID NO: 93 - DNA, 8 bp, Artificial Sequence
  Description of Artificial Sequence Synthetic oligonucleotide
actttagt

SEQ ID NO: 94 - DNA, 13 bp, Artificial Sequence
  Description of Artificial Sequence Synthetic oligonucleotide
tatctcgtaattg

SEQ ID NO: 95 - DNA, 20 bp, Artificial Sequence
  Description of Artificial Sequence Synthetic oligonucleotide
atatcatacccgagtagttg

SEQ ID NO: 96 - DNA, 10 bp, Artificial Sequence
  Description of Artificial Sequence Synthetic oligonucleotide
aggtctaact

SEQ ID NO: 97 - DNA, 14 bp, Artificial Sequence
  Description of Artificial Sequence Synthetic oligonucleotide
tatggttactacta

SEQ ID NO: 98 - DNA, 11 bp, Artificial Sequence
  Description of Artificial Sequence Synthetic oligonucleotide
gaacttgctta

SEQ ID NO: 99 - DNA, 19 bp, Artificial Sequence
  Description of Artificial Sequence Synthetic oligonucleotide
cagtggtaattccatattt

SEQ ID NO: 100 - DNA, 8 bp, Artificial Sequence
  Description of Artificial Sequence Synthetic oligonucleotide
tacagttt

SEQ ID NO: 101 - DNA, 18 bp, Artificial Sequence
  Description of Artificial Sequence Synthetic oligonucleotide
gagttaatttcacattcg

SEQ ID NO: 102 - DNA, 16 bp, Artificial Sequence
  Description of Artificial Sequence Synthetic oligonucleotide
gacctaacttcagggt

SEQ ID NO: 103 - DNA, 19 bp, Artificial Sequence
  Description of Artificial Sequence Synthetic oligonucleotide
ctaggcagttcatgcttat

SEQ ID NO: 104 - DNA, 15 bp, Artificial Sequence
  Description of Artificial Sequence Synthetic oligonucleotide
acctgctattaggtt

SEQ ID NO: 105 - DNA, 15 bp, Artificial Sequence
  Description of Artificial Sequence Synthetic oligonucleotide
ctcttcatggatatg

SEQ ID NO: 106 - DNA, 14 bp, Artificial Sequence
  Description of Artificial Sequence Synthetic oligonucleotide
cttacttgtggaac

SEQ ID NO: 107 - DNA, 11 bp, Artificial Sequence
  Description of Artificial Sequence Synthetic oligonucleotide
tttgacacttg

SEQ ID NO: 108 - DNA, 20 bp, Artificial Sequence
  Description of Artificial Sequence Synthetic oligonucleotide
gccgagtcagaattgttcac

SEQ ID NO: 109 - DNA, 12 bp, Artificial Sequence
  Description of Artificial Sequence Synthetic oligonucleotide
tctaatggcagc

SEQ ID NO: 110 - DNA, 12 bp, Artificial Sequence
  Description of Artificial Sequence Synthetic oligonucleotide
cgactatgtcag

SEQ ID NO: 111 - DNA, 12 bp, Artificial Sequence
  Description of Artificial Sequence Synthetic oligonucleotide
tcgacttcagga

SEQ ID NO: 112 - DNA, 20 bp, Artificial Sequence
  Description of Artificial Sequence Synthetic oligonucleotide
ctttgtgcaaagcgagtcac

SEQ ID NO: 113 - DNA, 15 bp, Artificial Sequence
  Description of Artificial Sequence Synthetic oligonucleotide
cgtatgtatcattgc

SEQ ID NO: 114 - DNA, 9 bp, Artificial Sequence
  Description of Artificial Sequence Synthetic oligonucleotide
ctcggaatt

SEQ ID NO: 115 - DNA, 10 bp, Artificial Sequence
  Description of Artificial Sequence Synthetic oligonucleotide
ccattttgag

SEQ ID NO: 116 - DNA, 11 bp, Artificial Sequence
  Description of Artificial Sequence Synthetic oligonucleotide
gcttctagtta

SEQ ID NO: 117 - DNA, 20 bp, Artificial Sequence
  Description of Artificial Sequence Synthetic oligonucleotide
aattacgccaaccttgggtg

SEQ ID NO: 118 - DNA, 16 bp, Artificial Sequence
  Description of Artificial Sequence Synthetic oligonucleotide
acctacgtttaggcga

SEQ ID NO: 119 - DNA, 18 bp, Artificial Sequence
  Description of Artificial Sequence Synthetic oligonucleotide
gcggaattttcactactg

SEQ ID NO: 120 - DNA, 17 bp, Artificial Sequence
  Description of Artificial Sequence Synthetic oligonucleotide
tggactaatcacggttc

SEQ ID NO: 121 - DNA, 8 bp, Artificial Sequence
  Description of Artificial Sequence Synthetic oligonucleotide
cgtcaatg

SEQ ID NO: 122 - DNA, 16 bp, Artificial Sequence
  Description of Artificial Sequence Synthetic oligonucleotide
atctgtgcaggtcaac

SEQ ID NO: 123 - DNA, 19 bp, Artificial Sequence
  Description of Artificial Sequence Synthetic oligonucleotide
cagtactctagggactttt

SEQ ID NO: 124 - DNA, 8 bp, Artificial Sequence
  Description of Artificial Sequence Synthetic oligonucleotide
ttaacgcg

SEQ ID NO: 125 - DNA, 13 bp, Artificial Sequence
  Description of Artificial Sequence Synthetic oligonucleotide
ttgtcagctagca

SEQ ID NO: 126 - DNA, 19 bp, Artificial Sequence
  Description of Artificial Sequence Synthetic oligonucleotide
cttctggagcgtctaatat

SEQ ID NO: 127 - DNA, 10 bp, Artificial Sequence
  Description of Artificial Sequence Synthetic oligonucleotide
gctaagttct

SEQ ID NO: 128 - DNA, 8 bp, Artificial Sequence
  Description of Artificial Sequence Synthetic oligonucleotide
tcgcagat

SEQ ID NO: 129 - DNA, 9 bp, Artificial Sequence
  Description of Artificial Sequence Synthetic oligonucleotide
gagtactct

SEQ ID NO: 130 - DNA, 8 bp, Artificial Sequence
  Description of Artificial Sequence Synthetic oligonucleotide
agtagcct

SEQ ID NO: 131 - DNA, 20 bp, Artificial Sequence
  Description of Artificial Sequence Synthetic oligonucleotide
tacactggttaggcatcacg

SEQ ID NO: 132 - DNA, 19 bp, Artificial Sequence
  Description of Artificial Sequence Synthetic oligonucleotide
ccagtgcatacgtttttga

SEQ ID NO: 133 - DNA, 15 bp, Artificial Sequence
  Description of Artificial Sequence Synthetic oligonucleotide
ctttctcgattagga

SEQ ID NO: 134 - DNA, 14 bp, Artificial Sequence
  Description of Artificial Sequence Synthetic oligonucleotide
actgactttgcatg

SEQ ID NO: 135 - DNA, 17 bp, Artificial Sequence
  Description of Artificial Sequence Synthetic oligonucleotide
cggccaattcgaatttg

SEQ ID NO: 136 - DNA, 12 bp, Artificial Sequence
  Description of Artificial Sequence Synthetic oligonucleotide
tcgcagatacgt

SEQ ID NO: 137 - DNA, 12 bp, Artificial Sequence
  Description of Artificial Sequence Synthetic oligonucleotide
gaacctgtagtc

SEQ ID NO: 138 - DNA, 19 bp, Artificial Sequence
  Description of Artificial Sequence Synthetic oligonucleotide
attagtatggtccctatgc

SEQ ID NO: 139 - DNA, 11 bp, Artificial Sequence
  Description of Artificial Sequence Synthetic oligonucleotide
gcattgttact

SEQ ID NO: 140 - DNA, 17 bp, Artificial Sequence
  Description of Artificial Sequence Synthetic oligonucleotide
atggatcgctcttacag

SEQ ID NO: 141 - DNA, 19 bp, Artificial Sequence
  Description of Artificial Sequence Synthetic oligonucleotide
gcatgtgccgtttctaata

SEQ ID NO: 142 - DNA, 17 bp, Artificial Sequence
  Description of Artificial Sequence Synthetic oligonucleotide
aacgcatcgtatcggtt

SEQ ID NO: 143 - DNA, 14 bp, Artificial Sequence
  Description of Artificial Sequence Synthetic oligonucleotide
ttgacgtccatgat

SEQ ID NO: 144 - DNA, 12 bp, Artificial Sequence
  Description of Artificial Sequence Synthetic oligonucleotide
ccagacttagtg

SEQ ID NO: 145 - DNA, 20 bp, Artificial Sequence
  Description of Artificial Sequence Synthetic oligonucleotide
atctgaagtggagcactctc

SEQ ID NO: 146 - DNA, 13 bp, Artificial Sequence
  Description of Artificial Sequence Synthetic oligonucleotide
gatcccgatatgt

SEQ ID NO: 147 - DNA, 19 bp, Artificial Sequence
  Description of Artificial Sequence Synthetic oligonucleotide
tactgtaggatgctctcta

SEQ ID NO: 148 - DNA, 16 bp, Artificial Sequence
  Description of Artificial Sequence Synthetic oligonucleotide
caactttcgcaaggtg

SEQ ID NO: 149 - DNA, 13 bp, Artificial Sequence
  Description of Artificial Sequence Synthetic oligonucleotide
gtctgcataatgc

SEQ ID NO: 150 - DNA, 12 bp, Artificial Sequence
  Description of Artificial Sequence Synthetic oligonucleotide
caatggtccatg

SEQ ID NO: 151 - DNA, 11 bp, Artificial Sequence
  Description of Artificial Sequence Synthetic oligonucleotide
tgcaatttcgt

SEQ ID NO: 152 - DNA, 15 bp, Artificial Sequence
  Description of Artificial Sequence Synthetic oligonucleotide
tatcgctattgctga

SEQ ID NO: 153 - DNA, 16 bp, Artificial Sequence
  Description of Artificial Sequence Synthetic oligonucleotide
tatgtcgcgagccaat

SEQ ID NO: 154 - DNA, 20 bp, Artificial Sequence
  Description of Artificial Sequence Synthetic oligonucleotide
ttgacacgtgaatgccactg

SEQ ID NO: 155 - DNA, 15 bp, Artificial Sequence
  Description of Artificial Sequence Synthetic oligonucleotide
gagcttcttattgac

SEQ ID NO: 156 - DNA, 16 bp, Artificial Sequence
  Description of Artificial Sequence Synthetic oligonucleotide
acgacttgatgtgcca

SEQ ID NO: 157 - DNA, 9 bp, Artificial Sequence
  Description of Artificial Sequence Synthetic oligonucleotide
ctatcgtga

SEQ ID NO: 158 - DNA, 17 bp, Artificial Sequence
  Description of Artificial Sequence Synthetic oligonucleotide
tcacgattagcttcagg

SEQ ID NO: 159 - DNA, 16 bp, Artificial Sequence
  Description of Artificial Sequence Synthetic oligonucleotide
tggcgatagactccta

SEQ ID NO: 160 - DNA, 15 bp, Artificial Sequence
  Description of Artificial Sequence Synthetic oligonucleotide
aattgcgtcttctag

SEQ ID NO: 161 - DNA, 14 bp, Artificial Sequence
  Description of Artificial Sequence Synthetic oligonucleotide
gttagtatccgtca

SEQ ID NO: 162 - DNA, 18 bp, Artificial Sequence
  Description of Artificial Sequence Synthetic oligonucleotide
tgatcgtaggtatctacc

SEQ ID NO: 163 - DNA, 10 bp, Artificial Sequence
  Description of Artificial Sequence Synthetic oligonucleotide
agtcttgtca

SEQ ID NO: 164 - DNA, 11 bp, Artificial Sequence
  Description of Artificial Sequence Synthetic oligonucleotide
ttcgtatgatc

SEQ ID NO: 165 - DNA, 13 bp, Artificial Sequence
  Description of Artificial Sequence Synthetic oligonucleotide
gcaggatctttca

SEQ ID NO: 166 - DNA, 8 bp, Artificial Sequence
  Description of Artificial Sequence Synthetic oligonucleotide
tcgacgta

SEQ ID NO: 167 - DNA, 16 bp, Artificial Sequence
  Description of Artificial Sequence Synthetic oligonucleotide
gatggagcattcccat

SEQ ID NO: 168 - DNA, 18 bp, Artificial Sequence
  Description of Artificial Sequence Synthetic oligonucleotide
tgatatacagtctgcgtc

SEQ ID NO: 169 - DNA, 9 bp, Artificial Sequence
  Description of Artificial Sequence Synthetic oligonucleotide
agtcacgtt

SEQ ID NO: 170 - DNA, 19 bp, Artificial Sequence
  Description of Artificial Sequence Synthetic oligonucleotide
agccgatctaggctatttt

SEQ ID NO: 171 - DNA, 14 bp, Artificial Sequence
  Description of Artificial Sequence Synthetic oligonucleotide
tagatctcactggt

SEQ ID NO: 172 - DNA, 9 bp, Artificial Sequence
  Description of Artificial Sequence Synthetic oligonucleotide
tgccagatt

SEQ ID NO: 173 - DNA, 10 bp, Artificial Sequence
  Description of Artificial Sequence Synthetic oligonucleotide
cgtctgatat

SEQ ID NO: 174 - DNA, 13 bp, Artificial Sequence
  Description of Artificial Sequence Synthetic oligonucleotide
gcttggacactta

SEQ ID NO: 175 - DNA, 17 bp, Artificial Sequence
  Description of Artificial Sequence Synthetic oligonucleotide
ccaggtagttcctagat

SEQ ID NO: 176 - DNA, 10 bp, Artificial Sequence
  Description of Artificial Sequence Synthetic oligonucleotide
tcgtgacatt

SEQ ID NO: 177 - DNA, 19 bp, Artificial Sequence
  Description of Artificial Sequence Synthetic oligonucleotide
ctctaccgatatgtgattg

SEQ ID NO: 178 - DNA, 9 bp, Artificial Sequence
  Description of Artificial Sequence Synthetic oligonucleotide
tagtcatgc

SEQ ID NO: 179 - DNA, 13 bp, Artificial Sequence
  Description of Artificial Sequence Synthetic oligonucleotide
tagctagactcgt

SEQ ID NO: 180 - DNA, 18 bp, Artificial Sequence
  Description of Artificial Sequence Synthetic oligonucleotide
ctttgaatgtagagctcc

SEQ ID NO: 181 - DNA, 18 bp, Artificial Sequence
  Description of Artificial Sequence Synthetic oligonucleotide
taggctgatttcagtacc

SEQ ID NO: 182 - DNA, 13 bp, Artificial Sequence
  Description of Artificial Sequence Synthetic oligonucleotide
attaagtctggcc

SEQ ID NO: 183 - DNA, 9 bp, Artificial Sequence
  Description of Artificial Sequence Synthetic oligonucleotide
tacgttcag

SEQ ID NO: 184 - DNA, 18 bp, Artificial Sequence
  Description of Artificial Sequence Synthetic oligonucleotide
gcgtcaatctgttcaatg

SEQ ID NO: 185 - DNA, 16 bp, Artificial Sequence
  Description of Artificial Sequence Synthetic oligonucleotide
acgtagaatgtctcgc

SEQ ID NO: 186 - DNA, 12 bp, Artificial Sequence
  Description of Artificial Sequence Synthetic oligonucleotide
gaagtgcttcca

SEQ ID NO: 187 - DNA, 11 bp, Artificial Sequence
  Description of Artificial Sequence Synthetic oligonucleotide
ttgagttctca

SEQ ID NO: 188 - DNA, 8 bp, Artificial Sequence
  Description of Artificial Sequence Synthetic oligonucleotide
tgcagtac

SEQ ID NO: 189 - DNA, 16 bp, Artificial Sequence
  Description of Artificial Sequence Synthetic oligonucleotide
ctctactgagcgatag

SEQ ID NO: 190 - DNA, 13 bp, Artificial Sequence
  Description of Artificial Sequence Synthetic oligonucleotide
ttagcgcagtcta

SEQ ID NO: 191 - DNA, 14 bp, Artificial Sequence
  Description of Artificial Sequence Synthetic oligonucleotide
gcttgacctttaag

SEQ ID NO: 192 - DNA, 17 bp, Artificial Sequence
  Description of Artificial Sequence Synthetic oligonucleotide
tgctatgaaggatccct

SEQ ID NO: 193 - DNA, 20 bp, Artificial Sequence
  Description of Artificial Sequence Synthetic oligonucleotide
ggcgccctatagagtactat

SEQ ID NO: 194 - DNA, 15 bp, Artificial Sequence
  Description of Artificial Sequence Synthetic oligonucleotide
aatatccgttcgttg

SEQ ID NO: 195 - DNA, 20 bp, Artificial Sequence
  Description of Artificial Sequence Synthetic oligonucleotide
gctcgtagtcgacaactgta

SEQ ID NO: 196 - DNA, 19 bp, Artificial Sequence
  Description of Artificial Sequence Synthetic oligonucleotide
ggcttagactttgtaatcc

SEQ ID NO: 197 - DNA, 18 bp, Artificial Sequence
  Description of Artificial Sequence Synthetic oligonucleotide
taattggatccaccgttg

SEQ ID NO: 198 - DNA, 9 bp, Artificial Sequence
  Description of Artificial Sequence Synthetic oligonucleotide
gactttcag

SEQ ID NO: 199 - DNA, 13 bp, Artificial Sequence
  Description of Artificial Sequence Synthetic oligonucleotide
catgtcctgtaga

SEQ ID NO: 200 - DNA, 8 bp, Artificial Sequence
  Description of Artificial Sequence Synthetic oligonucleotide
tgacagct

SEQ ID NO: 201 - DNA, 15 bp, Artificial Sequence
  Description of Artificial Sequence Synthetic oligonucleotide
caatgactttgttgc

SEQ ID NO: 202 - DNA, 20 bp, Artificial Sequence
  Description of Artificial Sequence Synthetic oligonucleotide
ctatgataatcaatgcattg

SEQ ID NO: 203 - DNA, 8 bp, Artificial Sequence
  Description of Artificial Sequence Synthetic oligonucleotide
ttcatgat

SEQ ID NO: 204 - DNA, 18 bp, Artificial Sequence
  Description of Artificial Sequence Synthetic oligonucleotide
ttgagttaatcactatat

SEQ ID NO: 205 - DNA, 14 bp, Artificial Sequence
  Description of Artificial Sequence Synthetic oligonucleotide
gttataaattcgtc

SEQ ID NO: 206 - DNA, 12 bp, Artificial Sequence
  Description of Artificial Sequence Synthetic oligonucleotide
ataatatttgct

SEQ ID NO: 207 - DNA, 17 bp, Artificial Sequence
  Description of Artificial Sequence Synthetic oligonucleotide
aaatttttgctttgaca

SEQ ID NO: 208 - DNA, 8 bp, Artificial Sequence
  Description of Artificial Sequence Synthetic oligonucleotide
acttatgt

SEQ ID NO: 209 - DNA, 19 bp, Artificial Sequence
  Description of Artificial Sequence Synthetic oligonucleotide
cttttataaatcgtagtat

SEQ ID NO: 210 - DNA, 20 bp, Artificial Sequence
  Description of Artificial Sequence Synthetic oligonucleotide
aagtcatcattgtacatagt

SEQ ID NO: 211 - DNA, 16 bp, Artificial Sequence
  Description of Artificial Sequence Synthetic oligonucleotide
actagttaatttgatc

SEQ ID NO: 212 - DNA, 10 bp, Artificial Sequence
  Description of Artificial Sequence Synthetic oligonucleotide
tactttagat

SEQ ID NO: 213 - DNA, 19 bp, Artificial Sequence
  Description of Artificial Sequence Synthetic oligonucleotide
aattcatatgcttagattt

SEQ ID NO: 214 - DNA, 12 bp, Artificial Sequence
  Description of Artificial Sequence Synthetic oligonucleotide
tagttacatatt

SEQ ID NO: 215 - DNA, 11 bp, Artificial Sequence
  Description of Artificial Sequence Synthetic oligonucleotide
aattttgttac

SEQ ID NO: 216 - DNA, 11 bp, Artificial Sequence
  Description of Artificial Sequence Synthetic oligonucleotide
tatatttgcat

SEQ ID NO: 217 - DNA, 14 bp, Artificial Sequence
  Description of Artificial Sequence Synthetic oligonucleotide
gattctgatatatc

SEQ ID NO: 218 - DNA, 11 bp, Artificial Sequence
  Description of Artificial Sequence Synthetic oligonucleotide
gatcattttat

SEQ ID NO: 219 - DNA, 18 bp, Artificial Sequence
  Description of Artificial Sequence Synthetic oligonucleotide
ttttataatcaggacatt

SEQ ID NO: 220 - DNA, 16 bp, Artificial Sequence
  Description of Artificial Sequence Synthetic oligonucleotide
ttcaatatctagtatg

SEQ ID NO: 221 - DNA, 12 bp, Artificial Sequence
  Description of Artificial Sequence Synthetic oligonucleotide
tatatttgatac

SEQ ID NO: 222 - DNA, 9 bp, Artificial Sequence
  Description of Artificial Sequence Synthetic oligonucleotide
catatgtta

SEQ ID NO: 223 - DNA, 13 bp, Artificial Sequence
  Description of Artificial Sequence Synthetic oligonucleotide
ttcttgataatat

SEQ ID NO: 224 - DNA, 16 bp, Artificial Sequence
  Description of Artificial Sequence Synthetic oligonucleotide
aagttattcttatcag

SEQ ID NO: 225 - DNA, 19 bp, Artificial Sequence
  Description of Artificial Sequence Synthetic oligonucleotide
attacgtactatttgatta

SEQ ID NO: 226 - DNA, 15 bp, Artificial Sequence
  Description of Artificial Sequence Synthetic oligonucleotide
ctctgattagaaatt

SEQ ID NO: 227 - DNA, 20 bp, Artificial Sequence
  Description of Artificial Sequence Synthetic oligonucleotide
tgccatttaataggatctaa

SEQ ID NO: 228 - DNA, 14 bp, Artificial Sequence
  Description of Artificial Sequence Synthetic oligonucleotide
attcttttacagag

SEQ ID NO: 229 - DNA, 16 bp, Artificial Sequence
  Description of Artificial Sequence Synthetic oligonucleotide
aagtatattttccatg

SEQ ID NO: 230 - DNA, 11 bp, Artificial Sequence
  Description of Artificial Sequence Synthetic oligonucleotide
atctattagtt

SEQ ID NO: 231 - DNA, 19 bp, Artificial Sequence
  Description of Artificial Sequence Synthetic oligonucleotide
ctttgataatgatttatac

SEQ ID NO: 232 - DNA, 13 bp, Artificial Sequence
  Description of Artificial Sequence Synthetic oligonucleotide
tagttaattctta

SEQ ID NO: 233 - DNA, 14 bp, Artificial Sequence
  Description of Artificial Sequence Synthetic oligonucleotide
ataatattcgtcgt

SEQ ID NO: 234 - DNA, 12 bp, Artificial Sequence
  Description of Artificial Sequence Synthetic oligonucleotide
gataattcttta

SEQ ID NO: 235 - DNA, 19 bp, Artificial Sequence
  Description of Artificial Sequence Synthetic oligonucleotide
attagttgtttcaaatcta

SEQ ID NO: 236 - DNA, 10 bp, Artificial Sequence
  Description of Artificial Sequence Synthetic oligonucleotide
tgcataattt

SEQ ID NO: 237 - DNA, 10 bp, Artificial Sequence
  Description of Artificial Sequence Synthetic oligonucleotide
atgtttcaat

SEQ ID NO: 238 - DNA, 10 bp, Artificial Sequence
  Description of Artificial Sequence Synthetic oligonucleotide
tagtttcata

SEQ ID NO: 239 - DNA, 18 bp, Artificial Sequence
  Description of Artificial Sequence Synthetic oligonucleotide
tgattaaatgtacatctt

SEQ ID NO: 240 - DNA, 19 bp, Artificial Sequence
  Description of Artificial Sequence Synthetic oligonucleotide
agactcttatttaagtatt

SEQ ID NO: 241 - DNA, 18 bp, Artificial Sequence
  Description of Artificial Sequence Synthetic oligonucleotide
attgaagttcttcattaa

SEQ ID NO: 242 - DNA, 8 bp, Artificial Sequence
  Description of Artificial Sequence Synthetic oligonucleotide
atttcatg

SEQ ID NO: 243 - DNA, 17 bp, Artificial Sequence
  Description of Artificial Sequence Synthetic oligonucleotide
tccttatatatgattga

SEQ ID NO: 244 - DNA, 18 bp, Artificial Sequence
  Description of Artificial Sequence Synthetic oligonucleotide
tataagttataactgctt

SEQ ID NO: 245 - DNA, 19 bp, Artificial Sequence
  Description of Artificial Sequence Synthetic oligonucleotide
ctctattaatatatagttg

SEQ ID NO: 246 - DNA, 8 bp, Artificial Sequence
  Description of Artificial Sequence Synthetic oligonucleotide
tcagttat

SEQ ID NO: 247 - DNA, 12 bp, Artificial Sequence
  Description of Artificial Sequence Synthetic oligonucleotide
atttatttacag

SEQ ID NO: 248 - DNA, 12 bp, Artificial Sequence
  Description of Artificial Sequence Synthetic oligonucleotide
atatattagtct

SEQ ID NO: 249 - DNA, 19 bp, Artificial Sequence
  Description of Artificial Sequence Synthetic oligonucleotide
attctgaatatgttacatt

SEQ ID NO: 250 - DNA, 10 bp, Artificial Sequence
  Description of Artificial Sequence Synthetic oligonucleotide
catattatgt

SEQ ID NO: 251 - DNA, 17 bp, Artificial Sequence
  Description of Artificial Sequence Synthetic oligonucleotide
ataattagctttgtcta

SEQ ID NO: 252 - DNA, 10 bp, Artificial Sequence
  Description of Artificial Sequence Synthetic oligonucleotide
tgttcttaaa

SEQ ID NO: 253 - DNA, 17 bp, Artificial Sequence
  Description of Artificial Sequence Synthetic oligonucleotide
tgttttaacattgacta

SEQ ID NO: 254 - DNA, 9 bp, Artificial Sequence
  Description of Artificial Sequence Synthetic oligonucleotide
ttgttaaca

SEQ ID NO: 255 - DNA, 11 bp, Artificial Sequence
  Description of Artificial Sequence Synthetic oligonucleotide
tcttttatgaa

SEQ ID NO: 256 - DNA, 14 bp, Artificial Sequence
  Description of Artificial Sequence Synthetic oligonucleotide
gtatcatttgctaa

SEQ ID NO: 257 - DNA, 12 bp, Artificial Sequence
  Description of Artificial Sequence Synthetic oligonucleotide
tctattaattag

SEQ ID NO: 258 - DNA, 11 bp, Artificial Sequence
  Description of Artificial Sequence Synthetic oligonucleotide
tatcttgttaa

SEQ ID NO: 259 - DNA, 10 bp, Artificial Sequence
  Description of Artificial Sequence Synthetic oligonucleotide
ttatcgaatt

SEQ ID NO: 260 - DNA, 12 bp, Artificial Sequence
  Description of Artificial Sequence Synthetic oligonucleotide
tgaactatttat

SEQ ID NO: 261 - DNA, 9 bp, Artificial Sequence
  Description of Artificial Sequence Synthetic oligonucleotide
ctaaagttt

SEQ ID NO: 262 - DNA, 18 bp, Artificial Sequence
  Description of Artificial Sequence Synthetic oligonucleotide
aatatagtcttcttgata

SEQ ID NO: 263 - DNA, 19 bp, Artificial Sequence
  Description of Artificial Sequence Synthetic oligonucleotide
aaggcaattttattcattt

SEQ ID NO: 264 - DNA, 9 bp, Artificial Sequence
  Description of Artificial Sequence Synthetic oligonucleotide
gttcaatta

SEQ ID NO: 265 - DNA, 20 bp, Artificial Sequence
  Description of Artificial Sequence Synthetic oligonucleotide
aagtttcgatattagaatcc

SEQ ID NO: 266 - DNA, 10 bp, Artificial Sequence
  Description of Artificial Sequence Synthetic oligonucleotide
tgtctataat

SEQ ID NO: 267 - DNA, 11 bp, Artificial Sequence
  Description of Artificial Sequence Synthetic oligonucleotide
ttcaattttga

SEQ ID NO: 268 - DNA, 13 bp, Artificial Sequence
  Description of Artificial Sequence Synthetic oligonucleotide
tttcattgaaatt

SEQ ID NO: 269 - DNA, 11 bp, Artificial Sequence
  Description of Artificial Sequence Synthetic oligonucleotide
aatgtctttat

SEQ ID NO: 270 - DNA, 20 bp, Artificial Sequence
  Description of Artificial Sequence Synthetic oligonucleotide
agaaattgaccattgtactt

SEQ ID NO: 271 - DNA, 19 bp, Artificial Sequence
  Description of Artificial Sequence Synthetic oligonucleotide
ttaactcgttttattagaa

SEQ ID NO: 272 - DNA, 20 bp, Artificial Sequence
  Description of Artificial Sequence Synthetic oligonucleotide
agatcatacgagcattattt

SEQ ID NO: 273 - DNA, 13 bp, Artificial Sequence
  Description of Artificial Sequence Synthetic oligonucleotide
aattttctaatgt

SEQ ID NO: 274 - DNA, 18 bp, Artificial Sequence
  Description of Artificial Sequence Synthetic oligonucleotide
ttatttgataccttgaaa

SEQ ID NO: 275 - DNA, 8 bp, Artificial Sequence
  Description of Artificial Sequence Synthetic oligonucleotide
ctagttta

SEQ ID NO: 276 - DNA, 14 bp, Artificial Sequence
  Description of Artificial Sequence Synthetic oligonucleotide
gactttcaagttta

SEQ ID NO: 277 - DNA, 11 bp, Artificial Sequence
  Description of Artificial Sequence Synthetic oligonucleotide
tcatgatttat

SEQ ID NO: 278 - DNA, 19 bp, Artificial Sequence
  Description of Artificial Sequence Synthetic oligonucleotide
tttaatttgttagaactca

SEQ ID NO: 279 - DNA, 20 bp, Artificial Sequence
  Description of Artificial Sequence Synthetic oligonucleotide
ctattttaggaaacacattg

SEQ ID NO: 280 - DNA, 14 bp, Artificial Sequence
  Description of Artificial Sequence Synthetic oligonucleotide
cgattatctgaatt

SEQ ID NO: 281 - DNA, 16 bp, Artificial Sequence
  Description of Artificial Sequence Synthetic oligonucleotide
aaaatcttagtcgttt

SEQ ID NO: 282 - DNA, 10 bp, Artificial Sequence
  Description of Artificial Sequence Synthetic oligonucleotide
ttacgattat

SEQ ID NO: 283 - DNA, 9 bp, Artificial Sequence
  Description of Artificial Sequence Synthetic oligonucleotide
ctatgatat

SEQ ID NO: 284 - DNA, 12 bp, Artificial Sequence
  Description of Artificial Sequence Synthetic oligonucleotide
aattattctagt

SEQ ID NO: 285 - DNA, 9 bp, Artificial Sequence
  Description of Artificial Sequence Synthetic oligonucleotide
tttagtaac

SEQ ID NO: 286 - DNA, 17 bp, Artificial Sequence
  Description of Artificial Sequence Synthetic oligonucleotide
gcgttttaatatacatt

SEQ ID NO: 287 - DNA, 9 bp, Artificial Sequence
  Description of Artificial Sequence Synthetic oligonucleotide
aaagttttc

SEQ ID NO: 288 - DNA, 14 bp, Artificial Sequence
  Description of Artificial Sequence Synthetic oligonucleotide
gtaacttcgttaat

SEQ ID NO: 289 - DNA, 13 bp, Artificial Sequence
  Description of Artificial Sequence Synthetic oligonucleotide
cttattaatagtt

SEQ ID NO: 290 - DNA, 14 bp, Artificial Sequence
  Description of Artificial Sequence Synthetic oligonucleotide
gatttacatttgac

SEQ ID NO: 291 - DNA, 12 bp, Artificial Sequence
  Description of Artificial Sequence Synthetic oligonucleotide
ttttaacattag

SEQ ID NO: 292 - DNA, 12 bp, Artificial Sequence
  Description of Artificial Sequence Synthetic oligonucleotide
ctaatttgatta

SEQ ID NO: 293 - DNA, 10 bp, Artificial Sequence
  Description of Artificial Sequence Synthetic oligonucleotide
tgtctaatta

SEQ ID NO: 294 - DNA, 16 bp, Artificial Sequence
  Description of Artificial Sequence Synthetic oligonucleotide
ttactttaaagttacg

SEQ ID NO: 295 - DNA, 12 bp, Artificial Sequence
  Description of Artificial Sequence Synthetic oligonucleotide
tatatgctttaa

SEQ ID NO: 296 - DNA, 19 bp, Artificial Sequence
  Description of Artificial Sequence Synthetic oligonucleotide
ttcttgatacatataagtt

SEQ ID NO: 297 - DNA, 13 bp, Artificial Sequence
  Description of Artificial Sequence Synthetic oligonucleotide
tatattcgatatt

SEQ ID NO: 298 - DNA, 16 bp, Artificial Sequence
  Description of Artificial Sequence Synthetic oligonucleotide
gtctcatgttttaaaa

SEQ ID NO: 299 - DNA, 17 bp, Artificial Sequence
  Description of Artificial Sequence Synthetic oligonucleotide
agttttaactgatcatt

SEQ ID NO: 300 - DNA, 9 bp, Artificial Sequence
  Description of Artificial Sequence Synthetic oligonucleotide
tgtatcata

SEQ ID NO: 301 - DNA, 10 bp, Artificial Sequence
  Description of Artificial Sequence Synthetic oligonucleotide
agttaatctt

SEQ ID NO: 302 - DNA, 20 bp, Artificial Sequence
  Description of Artificial Sequence Synthetic primer
gggcctgtcccagatatgtt

SEQ ID NO: 303 - DNA, 20 bp, Artificial Sequence
  Description of Artificial Sequence Synthetic primer
atgcctgaaaccgtaccaac

SEQ ID NO: 304 - DNA, 36 bp, Artificial Sequence
  Description of Artificial Sequence Synthetic probe
actgactaacaatagaaaatggttgggagggaatgg

SEQ ID NO: 305 - DNA, 20 bp, Artificial Sequence
  Description of Artificial Sequence Synthetic primer
tattccccaagccaagttca

SEQ ID NO: 306 - DNA, 20 bp, Artificial Sequence
  Description of Artificial Sequence Synthetic primer
cagcacgaggacttctttcc

SEQ ID NO: 307 - DNA, 37 bp, Artificial Sequence
  Description of Artificial Sequence Synthetic probe
actgactaacatcgaacaaaggtgtaacggcagcatg

SEQ ID NO: 308 - DNA, 20 bp, Artificial Sequence
  Description of Artificial Sequence Synthetic primer
cggcgaaagcttcaatactc

SEQ ID NO: 309 - DNA, 20 bp, Artificial Sequence
  Description of Artificial Sequence Synthetic primer
tctgtagggtcctcctggtg

SEQ ID NO: 310 - DNA, 37 bp, Artificial Sequence
  Description of Artificial Sequence Synthetic probe
actgactaacagacctgttacatccgggtgctttcct

SEQ ID NO: 311 - DNA, 20 bp, Artificial Sequence
  Description of Artificial Sequence Synthetic primer
gcgcagagactggaaagtgt

SEQ ID NO: 312 - DNA, 18 bp, Artificial Sequence
  Description of Artificial Sequence Synthetic primer
gcagtcctcgctcactgg

SEQ ID NO: 313 - DNA, 38 bp, Artificial Sequence
  Description of Artificial Sequence Synthetic probe
actgactaacattgaggctctcatggaatggctaaaga

SEQ ID NO: 314 - DNA, 20 bp, Artificial Sequence
  Description of Artificial Sequence Synthetic primer
gcaaggagccaatagaccag

SEQ ID NO: 315 - DNA, 20 bp, Artificial Sequence
  Description of Artificial Sequence Synthetic primer
gaagccgaatcctttgactc

SEQ ID NO: 316 - DNA, 38 bp, Artificial Sequence
  Description of Artificial Sequence Synthetic probe
actgaataacaactgacacgagcagaccatcagacaaa

SEQ ID NO: 317 - DNA, 19 bp, Artificial Sequence
  Description of Artificial Sequence Synthetic primer
attaggctttccgctggtg

SEQ ID NO: 318 - DNA, 20 bp, Artificial Sequence
  Description of Artificial Sequence Synthetic primer
ggtccgataaggggtcctac

SEQ ID NO: 319 - DNA, 38 bp, Artificial Sequence
  Description of Artificial Sequence Synthetic probe
actgactaacagttatcaatttgcccttggacaaggaa

SEQ ID NO: 320 - DNA, 18 bp, Artificial Sequence
  Description of Artificial Sequence Synthetic primer
ggccgcctcgtacaaaat

SEQ ID NO: 321 - DNA, 20 bp, Artificial Sequence
  Description of Artificial Sequence Synthetic primer
tgccagtgtctgggtaacag

SEQ ID NO: 322 - DNA, 38 bp, Artificial Sequence
  Description of Artificial Sequence Synthetic probe
actgactaacagagttgaatgcacccagttttcattat

SEQ ID NO: 323 - DNA, 20 bp, Artificial Sequence
  Description of Artificial Sequence Synthetic primer
ttcatgtggggcataaatca

SEQ ID NO: 324 - DNA, 20 bp, Artificial Sequence
  Description of Artificial Sequence Synthetic primer
ccctgctgaccattgacaag

SEQ ID NO: 325 - DNA, 37 bp, Artificial Sequence
  Description of Artificial Sequence Synthetic probe
actgactaacagccttcaaaccattgatagggccaag

SEQ ID NO: 326 - DNA, 21 bp, Artificial Sequence
  Description of Artificial Sequence Synthetic primer
aaccctagaactgtggggaag

SEQ ID NO: 327 - DNA, 18 bp, Artificial Sequence
  Description of Artificial Sequence Synthetic primer
gaggccctcccatccatt

SEQ ID NO: 328 - DNA, 37 bp, Artificial Sequence
  Description of Artificial Sequence Synthetic probe
actgactaacagttccagagaatccaaagcccagagg

SEQ ID NO: 329 - DNA, 20 bp, Artificial Sequence
  Description of Artificial Sequence Synthetic primer
catacaccctctcaccatcg

SEQ ID NO: 330 - DNA, 20 bp, Artificial Sequence
  Description of Artificial Sequence Synthetic primer
atctaccattccctgccatc

SEQ ID NO: 331 - DNA, 38 bp, Artificial Sequence
  Description of Artificial Sequence Synthetic probe
actgactaacaggagtgccccaaatatgtgaaatcaaa

SEQ ID NO: 332 - DNA, 20 bp, Artificial Sequence
  Description of Artificial Sequence Synthetic primer
gacgaattgctcacaaagca

SEQ ID NO: 333 - DNA, 20 bp, Artificial Sequence
  Description of Artificial Sequence Synthetic primer
gacgatcggtagccagagag

SEQ ID NO: 334 - DNA, 36 bp, Artificial Sequence
  Description of Artificial Sequence Synthetic probe
actgactaacacagcatccgtagccttattggcaac

SEQ ID NO: 335 - DNA, 22 bp, Artificial Sequence
  Description of Artificial Sequence Synthetic primer
aagcaacaaaaatgaaggcaat

SEQ ID NO: 336 - DNA, 21 bp, Artificial Sequence
  Description of Artificial Sequence Synthetic primer
tctgttgaattgttcgcatga

SEQ ID NO: 337 - DNA, 37 bp, Artificial Sequence
  Description of Artificial Sequence Synthetic probe
actgactaacagctatacacatttgcaaccgcaaatg

(19) United States
(12) Patent Application Publication
(10) Pub. No.: US 2014/0080725 A1
(43) Pub. Date: Mar. 20, 2014

(54) METHOD FOR SEPARATION AND DETECTION OF DNA FRAGMENTS

(71) Applicant: NANOMDX, INC., Northborough, MA (US)

(72) Inventor: Ranjit A. PRAKASH, Northborough, MA (US)

(73) Assignee: NANOMDX, INC., Northborough, MA (US)

(21) Appl. No.: 13/955,641

(22) Filed: Jul. 31, 2013

Related U.S. Application Data
(60) Provisional application No. 61/678,363, filed on Aug. 1, 2012.

(51) Int. Cl. C12Q 1/68

(52) U.S. Cl. CPC C12Q 1/6837
USPC 506/9

(57) ABSTRACT

A method of detecting nucleic acid fragments is provided. The method includes providing a plurality of chambers, each chamber separated from the other chambers of the plurality, each chamber having at least one set of probes disposed therein, each set of probes being capable of binding to a different target nucleic acid sequence relative to the other sets of probes. The method also includes providing a sample comprising a plurality of sets of nucleic acid fragments, placing at least a portion of the sample into each of the plurality of chambers and causing the at least one set of probes in each chamber to bind with complementary nucleic acid fragments of the sample, and detecting the binding of the nucleic acid fragments to the sets of probes in each chamber.

### CROSS-REFERENCE TO RELATED APPLICATIONS

This application claims the benefit of Application No. 61/678,363, entitled “Method for Separation and Detection of DNA Fragment,” filed Aug. 1, 2012, the contents of which are incorporated by reference herein.

This application is related to U.S. application Ser. No. TBA, entitled “Functionally Integrated Device for Multiplex Genetic Identification,” filed Jul. 31, 2013, Attorney Docket No. 2207797.121US2, and to U.S. application Ser. No. TBA, entitled “Enhanced Method for Probe Based Detection of Nucleic Acids,” filed Jul. 31, 2013, Attorney Docket No. 2207797.120US3, each of which is incorporated by reference herein.

### BACKGROUND OF THE INVENTION

1. Field of the Invention

The invention generally relates to methods for nucleic acid amplification, detection, and analysis of nucleotide molecules and sequences.

In addition, the invention generally relates to portable diagnostic tools and, more specifically, to biochip technology, which is also known as microfluidics or lab-on-a-chip technology.

2. Description of Related Art

When either detection method is applied to multiplex-PCR-amplified DNA fragments, similar size molecules will be indistinguishable (i.e., similar size molecules will migrate at identical speeds). Therefore, this size-dependence limitation requires that each unique multiplex-PCR-amplified DNA fragment should be of unique molecular size, and sufficiently unique to distinguish the fragments based on the separation resolution of the instrument. Furthermore, this size-dependence limitation also prevents capillary electrophoresis and mass spectroscopy methods from distinguishing between PCR-amplified DNA fragments featuring specifically-targeted DNA molecules or sequences and non-targeted PCR-amplified DNA fragments that may be of similar size.

Since the advent of nucleic acid-related technology decades ago, a number of methods have been developed for the detection and analysis of nucleic acid molecules (e.g., DNA). Some examples are capillary electrophoresis (including microfluidic electrophoresis), mass spectroscopy, southern blotting, and quantitative polymerase chain reaction (PCR), which may include real-time PCR methods and the use of TaqMan® probes (Roche Molecular Systems, Inc., Pleasanton, Calif.).

These detection methods have applications in, for example, in vitro DNA sequencing, gene expression quantification, genetic modification, genetic fingerprinting to identify a person or organism (e.g., for paternity testing, forensic science, and evolutionary studies), and diagnosis of disease (e.g., malignant cancers, hereditary diseases, and infectious agents). Often, the detection methods are paired with a PCR or similar nucleic acid amplification processes, which amplifies (i.e., replicates) the target DNA molecule or sequence in order to generate a sufficient amount of target DNA fragments to be detected. More recently, multiplex PCR was developed to amplify more than one unique target DNA molecule or sequence with a single PCR reaction.

In both capillary electrophoresis and mass spectroscopy, DNA molecules or sequences are separated and detected based on their molecular size/weight.

Quantitative or real-time PCR methods, often using TaqMan® probes, are widely used for the detection and analysis of PCR-amplified DNA fragments. A TaqMan® probe produces fluorescence when successfully bonded to a PCR-amplified DNA fragment. However, in a multiplex PCR reaction where more than one unique target DNA molecule or sequence is replicated, unique (i.e., differently colored) TaqMan® probes are necessary to distinguish the fluorescence from each of the DNA fragments. The signal sensitivity of and the capacity of the fluorescence detection instrument to distinguish the different fluorescent colors remain limiting factors, especially when the fluorescence emission spectra overlap.

### BRIEF SUMMARY OF THE INVENTION

The invention generally relates to improved methods using biochip technology for nucleic acid amplification, detection, and analysis of nucleotide molecules and sequences. Embodiments of the method use fluorescence to detect multiple DNA targets of similar or different size within a biochip by separating the DNA fragments into designated detection chambers in the biochip. Embodiments of the method may include providing a biochip having different separation and detection chambers, each with one or more probes, which have a complementary binding mechanism to a specific target DNA sequence; separating multiple DNA fragments in a sample into the biochip separation and detection chambers so that the DNA fragments bind, if at all, to a complementary probe; and using fluorescence to quantifiably detect the chamber-separated fragments. By separating different DNA fragments into these detection chambers, which are physically spaced apart from each other, embodiments improve the detection and analysis of target DNA sequences of similar size or with the same fluorescent label.

In some embodiments, a method of detecting nucleic acid fragments is provided. The method includes providing a plurality of chambers, each chamber separated from the other chambers of the plurality. Each chamber has at least one set of probes disposed therein, and each set of probes is capable of binding to a different target nucleic acid sequence relative to the other sets of probes. The method includes providing a sample including a plurality of sets of nucleic acid fragments and placing at least a portion of the sample into each of the plurality of chambers and causing the at least one set of probes in each chamber to bind with complementary nucleic acid fragments of the sample. The method also includes detecting the binding of the nucleic acid fragments to the sets of probes in each chamber.

In some embodiments, the placing the at least a portion of the sample into each of the plurality of chambers includes flowing the portions of the sample through a first chamber of the plurality of chambers. The chambers are fluidically coupled in series.

In other embodiments, the placing the at least a portion of the sample into each of the plurality of chambers comprises flowing the portions of the sample into the plurality of chambers. The chambers are fluidically coupled in parallel.

In some embodiments, the nucleic acid fragments are DNA fragments, and in other embodiments, the nucleic acid fragments are RNA fragments.

In some embodiments, the plurality of chambers are disposed in a biochip. The biochip includes an input port in fluid communication with the plurality of chambers.

In other embodiments, providing the sample includes performing a nucleic acid amplification. In some embodiments, the nucleic acid amplification includes at least one of PCR amplification and isothermal amplification.

In some embodiments, the sets of probes are fluorescently labeled, and the binding of the complementary nucleic acid fragments of the sample perseveres the fluorescence of the label. The detecting the binding of the nucleic acid fragments to the sets of probes includes detecting the fluorescence of the labels.

In some embodiments, the method includes quenching the fluorescent labels of unbound probes.

In some embodiments, the sets of probes are immobilized within the chambers.

In some embodiments, providing the sample comprises performing a nucleic acid amplification in which copies of amplified nucleic acid fragments are fluorescently labeled. The binding of the complementary nucleic acid fragments of the sample perseveres the fluorescence of the nucleic acid fragments. The detecting the binding of the nucleic acid fragments to the sets of probes includes detecting the fluorescence of the nucleic acid fragments.

In some embodiments, the method includes quenching the fluorescent labels of unbound nucleic acid fragments. In other embodiments, the method includes washing unbound nucleic acid fragments out of the plurality of plurality of chambers.

In some embodiments, providing the sample comprises performing a nucleic acid amplification in which copies of amplified nucleic acid fragments are amplified using fluorescently labeled bases of a specified type. At least one of the sets of probes has fluorescently labeled bases that are complementary to the specified type, the fluorescently labeled bases of the probes being quenched by binding to the fluorescently labeled bases of the nucleic acid fragments. The detecting the binding of the nucleic acid fragments to the sets of probes includes detecting when a fluorescently labeled base of the probes is not quenched.

### BRIEF DESCRIPTION OF THE DRAWINGS

For a more complete understanding of various embodiments of the present invention, reference is now made to the following descriptions taken in connection with the accompanying drawings in which:

FIG. 1 illustrates a biochip according to some embodiments;

FIG. 2 illustrates a separation and detection chamber that has been pre-coated with an immobilized probe according to some embodiments;

FIG. 3 illustrates a separation and detection chamber that has been pre-coated with different immobilized probes according to some embodiments;

FIG. 4 illustrates a flowchart of the method according to some embodiments;

FIG. 5 illustrates a diagram of options for fluorescence-based detection of complementary DNA fragment-probe binding according to some embodiments;

FIG. 6A illustrates a common quencher with a dye-quencher according to some embodiments;

FIG. 6B illustrates a probe containing a target specific sequence and a common quencher complementary sequence according to some embodiments;

FIG. 7A illustrates probes with a common quencher complementary sequence and target DNA fragments according to some embodiments;

FIG. 7B illustrates target DNA fragments bound to probes according to some embodiments;

FIG. 7C illustrates common quenchers bound to common quencher complementary sequences of probes according to some embodiments;

FIG. 8 illustrates an extended common quencher bound to a probe according to some embodiments;

FIG. 9 illustrates an increase in probe fluorescence for each of the twelve target DNA fragments by using the detection method according to some embodiments;

FIG. 10A lists forward primers, reverse primers, and probe sequences for the twelve targets; and

FIG. 10B lists two exemplary common quencher sequences with dye-quenching moieties according to embodiments of the present invention.

### DETAILED DESCRIPTION OF THE INVENTION

Embodiments of the invention provide an improved method of detecting and analyzing nucleotide sequences, which overcomes multiple limitations of existing methods by making it possible to differentiate nucleotide molecules or sequences of similar size or with the same fluorescent label. Some embodiments may be used to detect and analyze nucleotide molecules or sequences from the nucleic acids DNA and RNA. Hereinafter, a person of ordinary skill will understand that any references to DNA fragments or sequences would also apply more broadly to nucleotide molecules or sequences of another source.

Biochip technology offers numerous advantages for performing in vitro diagnostics, including the ability to integrate multiple biotechnology process steps in a single device, automate preprogrammed assays with minimal to no manual intervention, and enable portable diagnostic tools without the need for a large laboratory setup.

Embodiments of the present invention use fluorescence to detect multiple DNA targets of similar or different size within a biochip by separating the DNA fragments into designated detection chambers in the biochip. More specifically, embodiments include providing a biochip having different separation and detection chambers, each with one or more probes, which have a complementary binding mechanism to a specific target DNA sequence; separating multiple DNA fragments in a sample into the biochip separation and detection chambers so that the DNA fragments bind, if at all, to a complementary probe; and using fluorescence to quantifiably detect the chamber-separated fragments. By separating different DNA fragments into these detection chambers, which are physically spaced apart from each other, embodiments improve the detection and analysis of target DNA sequences. That is, DNA fragments of similar size or with the same fluorescent label can still be differentiated via separation into designated detection chambers in the biochip.

In alternative embodiments, the detection can be made without the biochip. Multiple vials or chambers contain unique probes that are either immobilized within the vial or retained in the vial, e.g., by being immobilized onto magnetic beads. When using the magnetic particles, a magnet can be energized above or below to retain the particles and the probes during fluid flow. For a sequential mechanism, the sample can be pipetted or input into vial 1 to cause binding of specific DNA to corresponding probes in vial 1. Then, the sample can be removed from vial 1, while the bound DNA-probe is retained in vial 1, and put into vial 2. The process can be repeated for the rest of the vials. For a parallel mechanism, the sample can be input into all of the vials and remove as needed.

Further embodiments of the biochip separation and detection method include first selectively amplifying one or more specific DNA sequences. PCR or isothermal amplification may be used to generate additional DNA fragments that are copies of a selected DNA sequence. Multiplex PCR may be used to select and replicate more than one unique DNA sequence at a time. Each amplified DNA fragment is itself a template for subsequent amplification. Thus, the target DNA sequence or sequences may be amplified exponentially, limited only by the available reagents and any feedback inhibition of amplified products, and the amplification process improves detection and analysis of DNA even from very small starting samples. Present embodiments allow these amplified DNA fragments to be of either unique or similar size. In addition, embodiments also allow these amplified DNA fragments to be labeled with either unique or identical fluorescent labels.

A person of ordinary skill will understand that various methods may be employed to amplify target DNA sequences. Preferred embodiments use a PCR or isothermal amplification reaction, combining a DNA sample with one or more DNA primers, nucleotides, a DNA polymerase, and various reagents known to a person of ordinary skill. For example, embodiments may be adjusted for buffers (e.g., Tris, Tricine, and Citrate), pH (e.g., 7 to 9), detergents (e.g., Tween), reducing agents (e.g., DTT), single-strand binding proteins, solvents (e.g., DMSO), salts (e.g., magnesium chloride, potassium chloride, and potassium acetate), derivatising agents (e.g., BSA), and bio stabilizers.

A DNA primer or oligonucleotide is a short DNA fragment containing a sequence complementary to the target DNA sequence. Two DNA primers may be used for each target DNA sequence, one primer that is complementary to the 3-prime end of the sense strand and one primer that is complementary to the 3-prime end of the antisense strand. Nucleotides containing triphosphate groups, i.e., deoxynucleoside triphosphates (dNTPs), may be assembled into new DNA fragments. A DNA polymerase enzymatically synthesizes new DNA fragments from dNTP by using each target DNA sequence template and the associated DNA primer. The DNA polymerase may be heat-stable, such as Taq polymerase. Preferred embodiments use native Taq or hot-start Taq polymerase. Also, in preferred embodiments, the target DNA sequence may range from 100 base pairs to 4 kilo base pairs.

A PCR reaction may consist of thermal cycling, that is, alternating cycles of heating and cooling the reaction according to a defined series of temperature steps. Alternatively, if isothermal amplification is used, a constant temperature may be maintained during the amplification process. The temperatures used and the time periods of application depend on, for example, the length of any target DNA sequences, the stability of the DNA polymerase, the melting temperatures of any DNA primers, and the concentrations of substrates and reagents. Specific embodiments may require additional temperature steps to be included at various points in the thermal cycle. The thermal cycle is repeated as desired or until the substrates and reagents are exhausted.

In embodiments where the DNA polymerase requires heat activation, the PCR reaction is initialized with a temperature of 95° C. for up to five minutes. The PCR reaction is then heated at 95° C. for five to forty-five seconds to disrupt the hydrogen bonds between complementary bases, thus achieving denaturation and physical separation of the two strands in the targeted DNA macromolecule in a process referred to as DNA melting (embodiments may be used to perform DNA melting curve analyses and discriminate between DNA sequences based on melting curve profiling in the presence of intercalating agents). The PCR reaction temperature is then lowered to a temperature ranging from 40° C. and 65° C. for five to forty-five seconds to allow DNA primers to anneal to the single-stranded templates of the target DNA sequence or sequences. In preferred embodiments, the melting temperature (Tm) of a DNA primer is less than 50° C. The temperature and timing is optimized for the DNA polymerase (e.g., 72° C. to 75° C. for five to forty-five seconds for Taq polymerase) to bind to the primer-template structure and synthesize a new DNA fragment complementary to the DNA template by adding dNTPs in the 5-prime to 3-prime direction. The timing of this DNA amplification step also depends on the length of the target DNA sequence or sequences.

Instead of thermal cycling through denaturation and amplification cycles, a helicase enzyme may be supplied during the PCR reaction to separate the two strands in the targeted DNA sample (or later in the process, to separate the stranded of the amplified DNA fragments for biochip probe binding).

Embodiments may amplify DNA molecules or fragments of similar size concurrently because the sizes of the amplified DNA fragments do not affect detection. Size independence is advantageous over existing DNA detection methods, such as capillary electrophoresis and mass spectroscopy, and enables optimal primer design for multiplex-PCR screening of genes that could result in similarly sized DNA fragments.

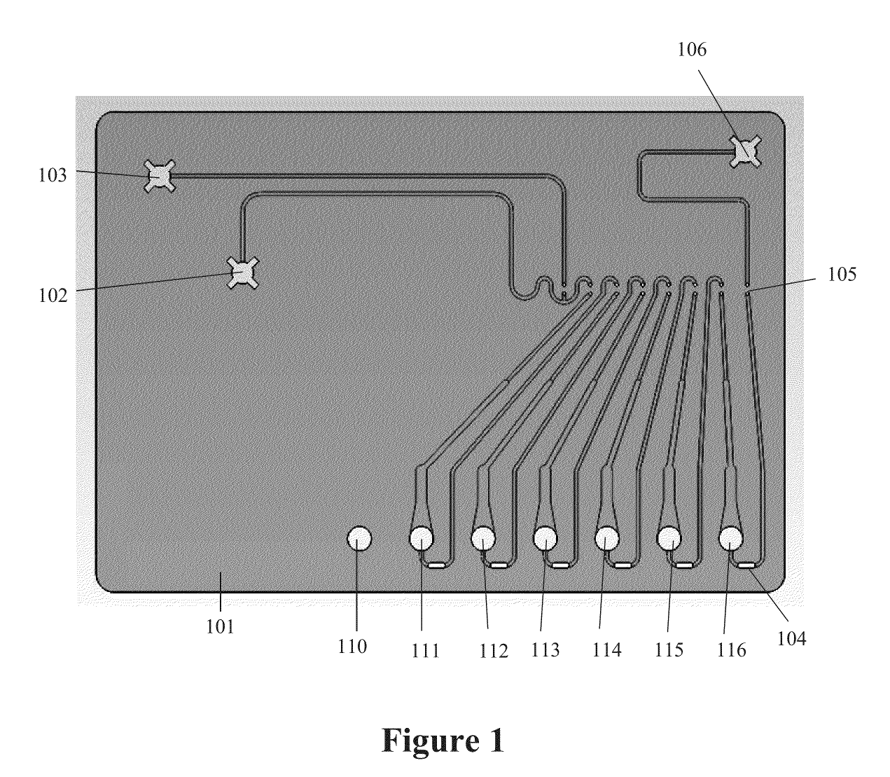

FIG. 1 depicts a biochip 101, which may be used according to some embodiments. Following FIG. 1, a multiplex-PCR sample is introduced to an input port 102 of a biochip 101. The multiplex-PCR-amplified DNA fragments are flowed through the biochip 101. The exemplary biochip in FIG. 1 features a separation wash buffer input port 103 and seven chambers. Chamber 110 has neither a probe nor fluid flow, and is used for differential background subtraction of fluorescence during the detection step. On the other hand, sequential separation and detection chambers 111 through 116 have been pre-coated with one or more immobilized probes that are designed to capture specific DNA fragments via complementary DNA-probe thermo-chemical interactions.

The DNA fragments are sequentially flowed through the series of biochip separation and detection chambers, beginning with the chamber 111. A vent membrane 104 enables the loading of the sample into each separation and detection chamber. The flow of the sample in the biochip is also aided by individual fluid flow gated controls 105. Any DNA fragments that are captured by a probe in the chamber 111 (i.e., bind to a specific complementary DNA sequence) remain bound to the probe while the remaining DNA fragments in the sample flow to the chamber 112. Likewise, any DNA fragments that are captured by a probe in the chamber 112 remain bound to the probe while the remaining DNA fragments in the sample flow to the chamber 113. In a similar manner, the flow continues through all six separation and detection chambers, one chamber at a time, resulting in the extraction and separation of DNA fragments into each designated chamber by a thermo-chemical DNA-probe binding. After passing through all of the separation and detection chambers, what remains of the sample flows to a waste unloading port 106. Alternatively, the separation and detection chambers can be in parallel such that the fluid can flow into a chamber without first flowing through another chamber. Detailed descriptions of the biochip is found in the incorporated application: “Functionally Integrated Device for Multiplex Genetic Identification.”

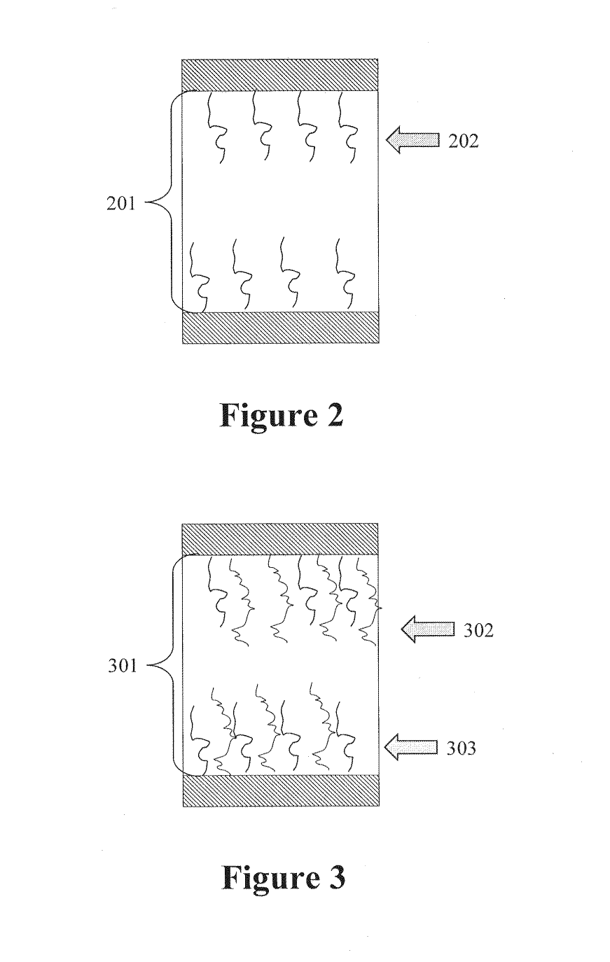

FIG. 2 illustrates, according to some embodiments, a separation and detection chamber 201 that has been pre-coated with an immobilized probe 202, which is designed to complementarily bind via thermo-chemical interaction to any DNA fragments featuring a specific target DNA sequence.

In preferred embodiments, a probe is immobilized directly to a surface of the biochip separation and detection chamber. A glass surface is preferred because glass has better studied binding chemistry and lower auto-fluorescence; however, the surface may be a plastic or similar material. In preferred embodiments, the Tm of an immobilized probe is greater than 75° C.

According to some embodiments, a probe is immobilized at its 5-prime end to a surface of a biochip separation and detection chamber. The immobilized probe has an amino linker at its 5-prime end and a fluorescent label (i.e., a fluorophore) bound to its free-floating 3-prime end. First, a fluorescence detection system is used to detect and quantify the fluorescence emission of the immobilized probe. Next, multiplex-PCR-amplified DNA fragments are introduced to the separation and detection chamber, and the chamber components are heated to 95° C. (to achieve DNA melting). The temperature is then ramped down to 45° C. to allow complementary DNA fragments (i.e., fragments featuring the target DNA sequence) to bind to the probe. A quencher oligonucleotide is included in the solution to quench the fluorescence emission of any immobilized probe that did not bind to a complementary DNA fragment by binding to that probe. A quencher oligonucleotide has a sequence that is complementary to the probe with a fluorescent label, so it can bind to the probe and quench the fluorescence. In preferred embodiments, the Tm of a quencher oligonucleotide is less than 45° C. Preferred embodiments may use one or more of the following quenchers: tetramethylrhodamine (TAMRA) or dihydrocyclopyrroloindole tripeptide (MGB). Following the binding process, at 45° C., the fluorescence detection system is again used to optically measure and quantify the fluorescence of the immobilized probe. A reduction in the fluorescence emission may indicate the absence or at least a low concentration of bound probe-DNA fragment pairs (and presumably the absence or at least a low concentration of the target DNA sequence in the sample).

According to some embodiments, a probe is immobilized at its 5-prime end to a surface of a biochip separation and detection chamber. The immobilized probe has an amino linker and a quencher oligonucleotide at its 5-prime end while a fluorescent label (i.e., a fluorophore) is bound to its free-floating 3-prime end. The first ten bases from each end of the immobilized probe are complementary to each other, and the immobilized probe is designed to bind complementary DNA fragments in the region between its 5-prime end and 3-prime end (excluding the ten bases from each end). First, a fluorescence detection system is used to detect and quantify the fluorescence emission of the immobilized probe. Next, multiplex-PCR-amplified DNA fragments are introduced to the separation and detection chamber, and the chamber components are heated to 95° C. (to achieve DNA melting). The temperature is then ramped down to 45° C. to allow complementary DNA fragments (i.e., fragments featuring the target DNA sequence) to bind to the probe. If the immobilized probe does not bind to a complementary DNA fragment, then the probe will collapse and bind to itself (i.e., the 3-prime end will bind to the 5-prime end), quenching the fluorescence of the probe. Following the binding process, at 45° C., the fluorescence detection system is again used to optically measure and quantify the fluorescence of the immobilized probe. A reduction in the fluorescence emission indicates the absence or at least a low concentration of bound probe-DNA fragment pairs (and presumably the absence or at least a low concentration of the target DNA sequence in the sample).

According to some embodiments, a probe is immobilized at its 5-prime end to a surface of a biochip separation and detection chamber while its 3-prime end is free-floating. The immobilized probe is not fluorescently labeled with a fluorophore. Instead, the DNA primers have fluorescent labels. Thus, the multiplex-PCR-amplified DNA fragments are fluorescently labeled during amplification. Once the DNA fragments are introduced to the separation and detection chamber, complementary DNA fragments (i.e., fragments featuring the target DNA sequence) bind to the probe. The binding process is aided by the presence of a chemical such as 2×SSC (sodium chloride and sodium citrate solution) and a constant temperature between 40° C. and 60° C. Unbound DNA fragments may be washed out with a separation wash buffer. Alternatively, a quencher oligonucleotide may be included in the solution to quench the fluorescence emission of any DNA fragment that did not bind to the probe. In this case, a quencher oligonucleotide can have a sequence complementary to the DNA fragments, so it can bind to the DNA fragments and quench the fluorescence. A fluorescence detection system is used to optically measure and quantify the fluorescence of the bound probe-DNA fragment pairs. The binding of all probes in the separation and detection chamber will result in a maximal fluorescent signal.

According to some embodiments, a probe is immobilized at its 5-prime end to a surface of a biochip separation and detection chamber while its 3-prime end is free-floating. The immobilized probe has a fluorescent label (i.e., a fluorophore) present in one of its bases (e.g., G or guanine) Meanwhile, in the PCR reaction, dNTPs with a complementary base (e.g., C or cytosine) are also fluorescently labeled. Once the multiplex-PCR-amplified DNA fragments are introduced to the separation and detection chamber, complementary DNA fragments (i.e., fragments featuring the target DNA sequence) bind to the immobilized probe. The fluorescent label in the immobilized probe is quenched if and only if complementary binding occurs (e.g., fluorescently-labeled base G in an immobilized probe is quenched by fluorescently-labeled base C in a DNA fragment). This embodiment is particularly useful for detection of single-nucleotide polymorphism (SNP).

Embodiments may result in double specificity for PCR-amplified DNA fragments. During amplification, DNA primers are designed to be specifically complementary to a target DNA sequence. Then, during space separation, the amplified DNA fragments are bound to specifically complementary probes, if they exist, in the biochip separation and detection chambers. This double specificity increases the ability to discriminate between target and non-target PCR-amplified DNA fragments. Double specificity is advantageous over existing DNA detection methods, such as capillary electrophoresis and methods that rely solely on micro-arrays.

A person of ordinary skill will understand that various methods may be employed to fluorescently label macromolecules, such as DNA fragments and probes, according to certain embodiments. Preferred embodiments use fluorophores (e.g., TaqMan® probes), which absorb light energy of a specific wavelength and re-emit light at a longer wavelength. In combination with a fluorescence detection system, a fluorophore may indicate the presence (or absence) of a specific nucleotide molecule or sequence and the concentration thereof. For example, an embodiment may be designed to result in fluorescence only when there is a successful DNA fragment-probe complementary binding. Alternatively, a fluorescence reduction method is designed to result in quenching (i.e., loss of fluorescent emission) only when there is a successful DNA fragment-probe complementary binding.

Fluorophores may differ in their maximum excitation wavelength, maximum emission wavelength, extinction coefficient, quantum yield, lifetime, and other properties. Preferred embodiments may use one or more of the following: EvaGreen® (Biotium, Inc., Hayward, Calif.), TYE™ (Integrated DNA Technologies, Inc., Coralville, Iowa), FAM™ (Applera Corp., Norwalk, Conn.), VIC® (Applera Corp.), TET™ (Applied Biosystems, Inc., Foster City, Calif.), ROX™ (Applied Biosystems, Inc.), SYBR® Green (Molecular Probes, Inc., Eugene, Oreg.), and Alexa Fluor® dyes (Molecular Probes, Inc.).

In preferred embodiments, after the different types of DNA fragments have been separated into designated biochip detection chambers according to a specific target DNA sequence, a fluorescence detection system is used to detect the fluorescence of the immobilized probe-DNA fragment pairs in each of the separation and detection chambers.

Fluorescence detection can be accomplished either with or without a separation wash buffer. However, immobilization of any probes may be necessary if a wash buffer is used; otherwise any probes in the separation and detection chambers may be washed out.

A person of ordinary skill will understand numerous options for fluorescence detection. For example, a fluorometer or spectrofluorometer may be used to measure the parameters of fluorescence, including the intensity and wavelength distribution of light emission spectra after excitation by a certain spectrum of light. Possible light sources that provide excitation energy capable of inducing fluorescence (usually fluorescent light in the wavelength range of 350 nm to 900 nm, comprising blue, green, and red wavelength spectra) include a laser, a photodiode, a mercury-vapor lamp, and a xenon arc lamp. Thus, these parameters may be used to identify the presence or absence as well as the amount of immobilized probe-DNA fragment pairs in each of the separation and detection chambers.

In some embodiments, a fluorometer uses two light beams to counteract signal noise produced by radiant power fluctuations. An incident light beam is filtered and passed through the sample, which absorbs the light then emits fluorescence as it returns to a lower energy state. A second beam is attenuated and adjusted to match the intensity of the fluorescence emitted by the sample. Separate transducers detect the second beam and the fluorescent emission from the sample, converting each to electrical signals for interpretation by a computer system. In other embodiments, the fluorescent emission passes through a second filter or monochromator, which is placed at 90° to the incident light beam to minimize the risk of transmitted or reflected incident light reaching the transducer. An additional way to counteract signal noise, according to some embodiments, is to include a base sample for differential background subtraction of fluorescence, such as biochip chamber 0 in FIG. 1.

Fluorescence detection is limited by the color detection capability of the fluorescence detection system (i.e., the ability of the instrument to distinguish the different fluorescent colors). For example, a single-color fluorometer can detect only one fluorescent label, while a three-color fluorometer can detect up to three unique fluorescent labels. Typically, when multiple fluorescent labels are used (especially more than the three clearly distinguished blue, green, and red spectra), the light emission spectra may overlap each other, making it difficult to distinguish the unique fluorescent labels. Software algorithms may be employed to compensate for overlapping emission spectra; however, signal sensitivity may nevertheless be compromised. Currently, the best available multi-color fluorescence detection system identifies up to nine colors, with wavelengths ranging from about 350 nm to about 950 nm, a range which includes blue, green, and red light emission spectra.

In preferred embodiments, because the biochip separation and detection chambers are physically separated, a fluorescence detection system may be used to detect the emission spectra from each of the separation and detection chambers without interference from the emission spectra in other separation and detection chambers. Thus, a single-color fluorometer may be used to detect the fluorescence of the immobilized probe-DNA fragment pairs in each of the separation and detection chambers independent of size and fluorescent label.

FIG. 3 illustrates, according to some embodiments, a separation and detection chamber 301 that has been pre-coated with one immobilized probe 302 and one different immobilized probe 303, the two of which are designed to bind to two different target DNA sequences. For ease of detection and analysis, the two different immobilized probes are designed, in some embodiments, to emit different fluorescent colors upon binding to DNA fragments in the sample. However, a multi-color fluorometer must be available.

For example, if a nine-color fluorescence detection system is available, then theoretically the immobilized probe-DNA fragment pairs in each separation and detection chamber may be labeled with as many as nine different colors, representing up to nine different target DNA sequences. If a biochip has six separation and detection chambers, as in FIG. 1, and nine different immobilized probes in each chamber, then an embodiment may be capable of detecting up to fifty-four different target DNA sequences.

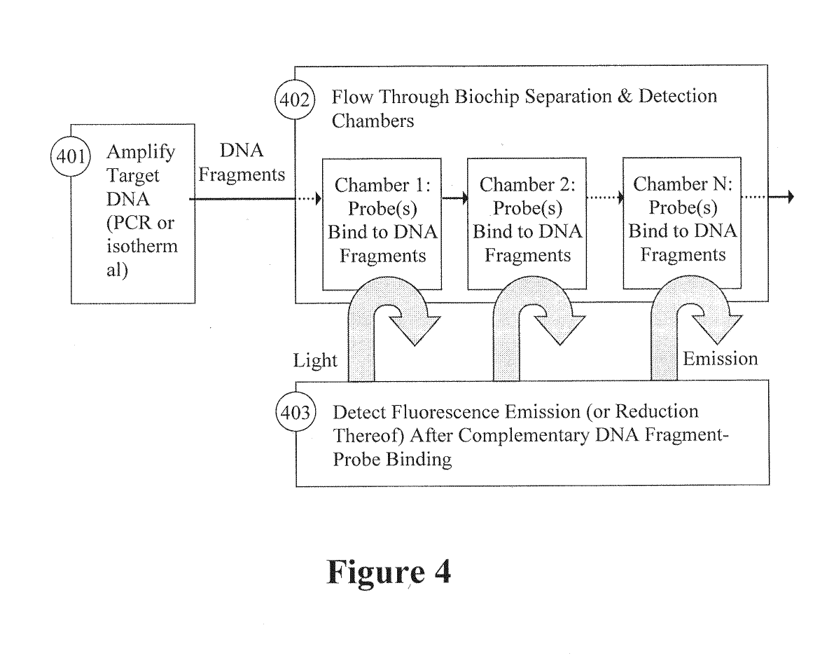

FIG. 4 illustrates a flowchart of the method according to some embodiments. In step 401, target DNA is amplified (e.g., by PCR or isothermal amplification). In step 402, the resulting DNA fragments are flowed sequentially through N separation and detection chambers of a biochip. In each separation and detection chamber, immobilized probes bind to any complementary DNA fragments, and any unbound DNA fragments flow to the next chamber until the Nth chamber. Following the separation of DNA fragments via DNA fragment-probe thermo-chemical interactions in the detection chambers, in step 403, the fluorescence emissions, or reduction thereof, is measured and analyzed.

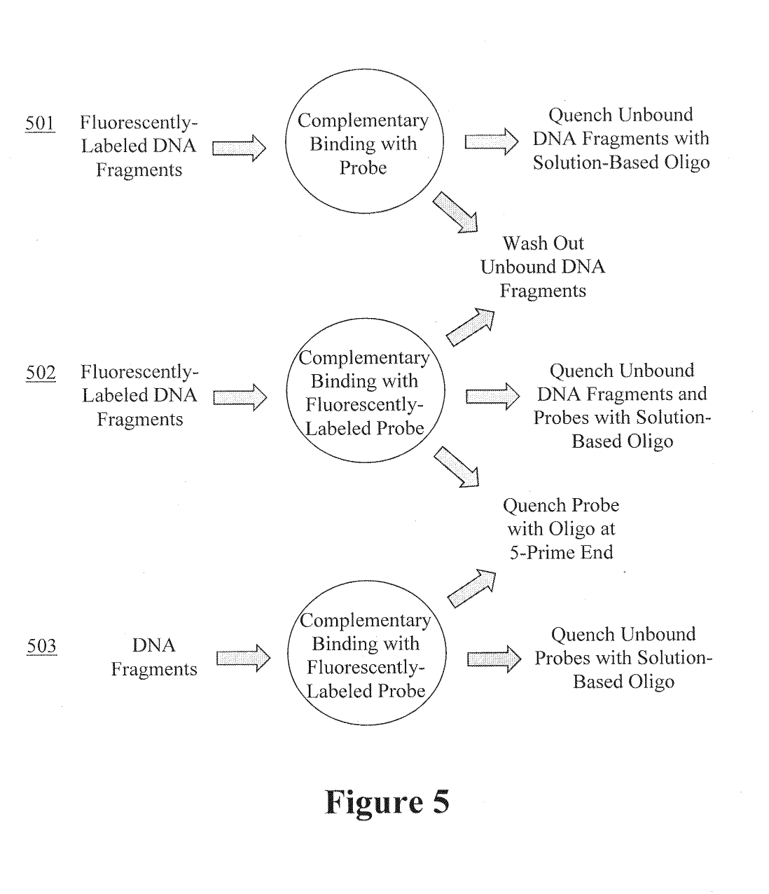

FIG. 5 illustrates a diagram of options for fluorescence-based detection of complementary DNA fragment-probe binding according to some embodiments. In case 501, a fluorescently-labeled DNA fragment binds with a probe in a separation and detection chamber. In order to detect only the complementary DNA fragment-probe pair and not any unbound DNA fragments, which also may be fluorescently labeled, several steps may be taken. First, according to some embodiments, a quencher oligonucleotide may be included in the solution to quench the fluorescence emission of any unbound DNA fragments. Second, according to embodiments with immobilized probes, the separation and detection chamber may be flushed of any unbound DNA fragments with a wash buffer.

In case 502, a fluorescently-labeled DNA fragment binds with a fluorescently-labeled probe in a separation and detection chamber. Assuming complementary binding quenches the fluorescence emissions of both the DNA fragment and the probe, a reduction in fluorescence emissions from the chamber may indicate the complementary DNA fragment-probe pair. However, any unbound DNA fragments and any unbound probes still may be fluorescently labeled. Again, according to some embodiments, a quencher oligonucleotide may be included in the solution to quench the fluorescence emission of any unbound DNA fragments or, in this case, any unbound probes. According to embodiments with immobilized probes, the separation and detection chamber may be flushed of any unbound DNA fragments with a wash buffer; however, any unbound and fluorescently-labeled probes will stay immobilized in the chamber. According to embodiments with probes having a quencher oligonucleotide at the 5-prime end, any unbound probes may self-quench by collapsing and binding 5-prime end to 3-prime end.

In case 503, a DNA fragment binds with a fluorescently-labeled probe in a separation and detection chamber. In order to detect only the complementary DNA fragment-probe pair and not any unbound probes, according to some embodiments, a quencher oligonucleotide may be included in the solution to quench the fluorescence emission of any unbound probes. Also, according to embodiments with probes having a quencher oligonucleotide at the 5-prime end, any unbound probes may self-quench by collapsing and binding 5-prime end to 3-prime end.

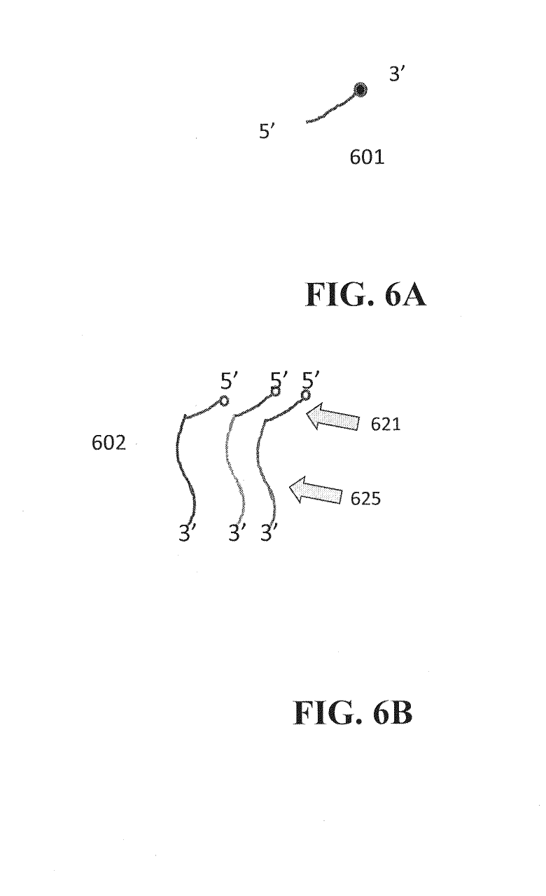

In cases 502 and 503, a common quencher method can be used. A set of probes has a specified sequence (a common quencher complementary sequence 621) in addition to a target specific sequence 625 capable of binding a target fragment. Each probe of the set of probes has the same common quencher complementary sequence 621 and a different gene specific sequence 625 depending on the target sequence. A quenching compound (or a common quencher) has the same common quencher sequence and a dye-quenching moiety attached to the sequence. Because the probes use the same common quencher complementary sequence 621, quenching compounds having one type of common quencher sequence—complementary to sequence 621—can quench fluorescence of all of the probes. FIG. 6A shows a common quencher 601, which contains a 3-prime end quencher with a dye-quencher (or a dye-quenching moiety). FIG. 6B shows a probe 602 containing a gene specific sequence 625 and a common quencher complementary sequence 621 at the 5-prime end. The 5-prime end of the probe is fluorescent labeled. A common quencher sequence is a random DNA sequence of 8-20 bp lengths. A non-exhaustive examples of over 300 such sequences designed by NanoMDx are attached in the DNA sequence listing, provided with the application. The sequence listing includes 3 generated lists, each list containing 100 sequences. The list can be combined to one list containing 300 sequences.

Using a common quencher method, each probe 602 with target specific sequence 625 used for multiple DNA target detection can have complementary sequence 621 to the common quencher. This configuration of probe allows binding of a common quencher oligonucleotide to the probe containing a complementary quencher sequence. Thus, one or few common quencher oligonucleotides can be used for a plurality of probes designed to bind to their corresponding DNA targets. When a common quencher binds to the probe, the dye-quenching moiety of the common quencher will quench the fluorescence of the probe. Similar to target specific quenchers, a common quencher oligonucleotide and probe will bind to each other under certain conditions (e.g., when the temperature is below 45° C.).

Some randomly generated common quencher sequences may have sequences complimentary to the target specific sequences 625 or to the target DNA sequences. To avoid conflicts of sequences (i.e., common quenchers binding to sequences other than complementary sequences 621), some common quencher sequences are avoided in some embodiments. Alternatively, more than one common quencher sequences are used to quench every unbound probe.

The common quencher oligonucleotide and many target specific probes can be mixed together to form the final probe for the detection process. The probe and the common quencher are bound together to constitute the PCR mix.

This method of using only one or a few ‘common quenchers’ for all the detection probes in a multiplex-PCR reaction is cost saving and simplifies the overall detection process. Conventionally, a separate quencher is required for each detection probe/PCR product, such as those used in TaqMan® assay and other real-time PCR approaches.

The following is an exemplary, not exhaustive, list of salient features of a common quencher based detection methods. The length of a target specific oligonucleotide ranges from about 15 bp to about 45 bp. The melting temperature of a target specific oligonucleotide ranges from about 45° C. to about 75° C. The guanine-cytosine percentage (GC %) of a target oligonucleotide ranges from about 25% to about 45%. A target specific oligonucleotide has a sequence complementary to the intended target and a sequence complementary to common oligonucleotide. A target specific oligonucleotide can also have 3-terminal modification enabling it to bind to solid surface. The 5-prime end of target specific oligonucleotide will have a fluorescent dye either Cy5, Tye, EvaGreen®, TYE™, FAM™, VIC®, TET™, ROX™, and Alexa Fluor® dyes. The length of common oligonucleotide can range from about 8 bp to about 20 bp. The melting temperature of common oligonucleotide ranges from about 25° C. to about 45° C. The GC % of common oligonucleotide ranges from about 5% to about 30%. The 3-prime end of common oligonucleotide has a quencher dye attached. A single common oligonucleotide quenches fluorescence from all other target specific oligonucleotides. A target specific oligonucleotide binds to a specific target when the temperature is about 45° C. to about 75° C. A common oligonucleotide binds to target specific oligonucleotide when the temperature is about 25° C. to about 45° C. At this temperature, free unbound target specific oligonucleotides will be hybridized to common oligonucleotides. Fluorescence from a target specific probe in the presence of intended targets and common oligonucleotides are measured at about 30° C. Binding between target specific oligonucleotides and intended targets and between target specific oligonucleotides and the common oligonucleotides are performed by ramping up the endpoint PCR or isothermal amplification reaction mix from about 25° C. to about 95° C., and ramp-down from about 95° C. to about 25° C. Binding between target specific oligonucleotides and the intended targets and between target specific oligonucleotides and common oligonucleotides are performed by incubating the PCR or isothermal amplification reaction mix at about 60° C. followed by incubation at about 25° C. The common quencher method can be applied to both real-time and end-point PCR amplifications. The design of target specific oligonecleotides and common oligonucleotides can be used as an endpoint detection method after amplification or as a real-time PCR application detection method.

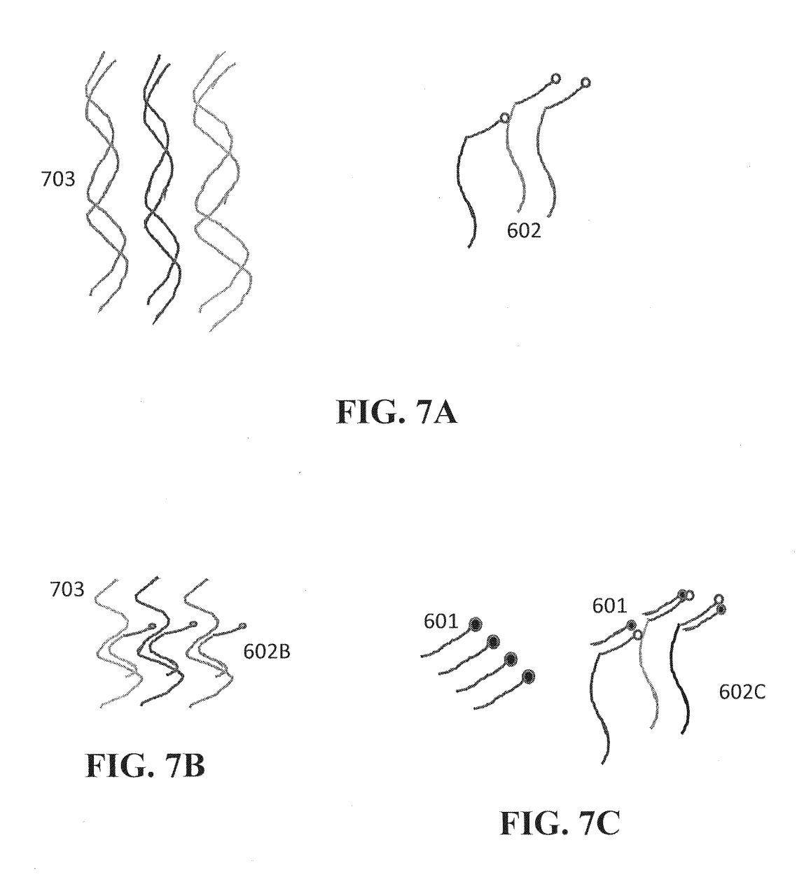

Similar to the use of target specific probes, FIGS. 7A, 7B, and 7C illustrate a fluorescent detection process using the common quenching method according to some embodiments. Amplified DNA fragments 703 are provided to chambers containing probes 602 with common quencher complementary sequences of FIG. 7A provided into chambers. After the DNA fragments are provided, the temperature is raised to 95° C. to achieve DNA melting and ramped down to 45° C. to allow complementary DNA fragments to bind to the probe. FIG. 7B illustrates the DNA fragments 703 bound to probes 602B. For the remaining probes 602C that do not have binding DNA fragments, common quenchers 601 quenches the probe fluorescence, as illustrated in FIG. 7C. Then, the fluorescence from the DNA fragments can be observed.

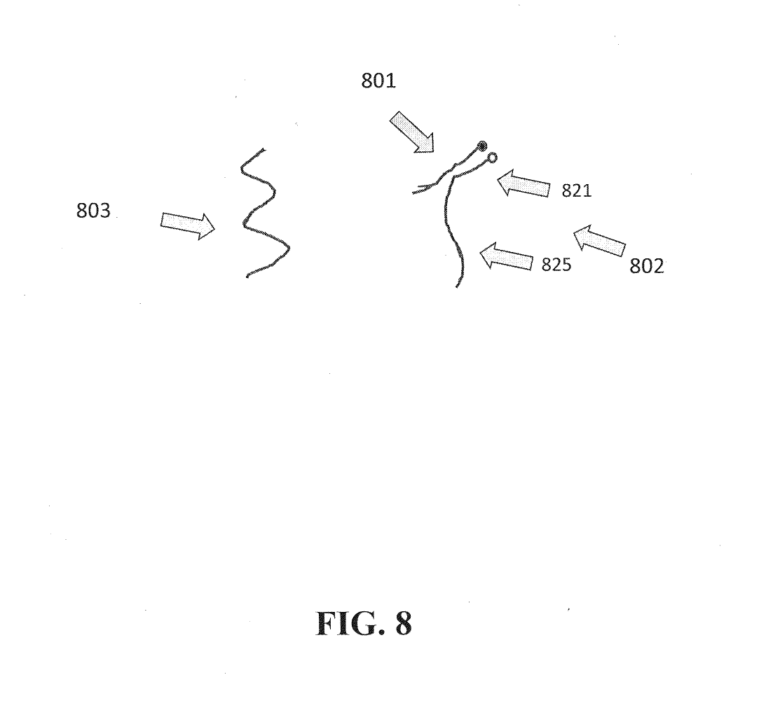

In some embodiments, a common quencher 801 can be designed slightly longer than a common quencher complementary sequence 821 as shown in FIG. 8. In this case, after a common quencher 801 binds to the common quencher complementary sequence 821 of a probe 802, the extending portion of the common quencher 801 increases the steric hindrance, thereby decreasing the likelihood that a target DNA 803 would bind to the target specific complementary sequence 825 of the probe 802.

Four examples are described below. In Examples 1 and 2, separation and detection chambers are in series. Thus, fluid sequentially flows from one channel to another channel. Also, in Examples 1 and 2, probes are immobilized to the surface or beads in the chamber. In Examples 3 and 4, separation and detection chambers are in parallel. Since fluid does not flow sequentially through all of the separation and detection chambers, a wash buffer is optional. Thus, the probes do not need to be immobilized. Examples 3 and 4 shows the testing results with different numbers of colors and targets.

### Example 1

In a first exemplary embodiment, a biochip with six separation and detection chambers is used with a single-color fluorescence detection system to simultaneously detect as many as six target DNA sequences. A multiplex PCR or isothermal amplification process is used to generate a sample comprising six unique types of DNA fragments, which are of similar or different size. All six types of DNA fragments are labeled with an identical fluorescent color.

The sample is introduced to an input port of the biochip and flowed sequentially through a series of six separation and detection chambers, which have each been pre-coated with an immobilized probe designed to bind to a specific target DNA sequence. Chamber 1 has been pre-coated with immobilized probe A, which was designed to complementarily bind via thermo-chemical interaction, to any DNA fragments featuring target DNA sequence A. When the sample comprising unique DNA fragment types 1-6 are first loaded into chamber 1, and if one of the fragment types features target DNA sequence A, all of the DNA fragments of that type are captured, essentially extracted from the sample, and retained in chamber 1. The rest of the sample, including the other DNA fragments, is flowed to chamber 2, thus physically separating any DNA fragments featuring target DNA sequence A from the rest of the sample.

Chamber 2 has been pre-coated with immobilized probe B, which was designed to complementarily bind via thermo-chemical interaction, to any DNA fragments featuring target DNA sequence B. If one of the fragment types features target DNA sequence B, all of the DNA fragments of that type are captured, essentially extracted from the sample, and retained in chamber 2. The rest of the sample is flowed to chamber 3, thus physically separating any DNA fragments featuring target DNA sequence B from the rest of the sample. In a similar manner, the sample is flowed through the remaining four separation and detection chambers, one chamber at a time, resulting in the extraction and physical separation of DNA fragments into the six separation and detection chambers according to the presence of target DNA sequences.

Following space separation, the single-color fluorescence detection system is used to detect the fluorescence emission from each biochip separation and detection chamber, which contains paired immobilized probes and DNA fragments. Because the unique types of DNA fragments have been physically separated according to target DNA sequence, all six types of DNA fragments may be labeled with an identical fluorescent color and still be detected by a single-color fluorescence detection system.

### Example 2

In a second exemplary embodiment, a biochip with six separation and detection chambers is used with a three-color fluorescence detection system to simultaneously detect as many as eighteen target DNA sequences. A multiplex PCR or isothermal amplification process is used to generate a sample comprising eighteen unique types of DNA fragments, which are of similar or different size. Six types of DNA fragments are labeled with a first fluorescent color, six other types of DNA fragments are labeled with a second fluorescent color, and the remaining six types of DNA fragments are labeled with a third fluorescent color.

The sample is introduced to an input port of the biochip and flowed sequentially through a series of six separation and detection chambers, which have each been pre-coated with three different immobilized probes, each designed to bind to specific target DNA sequences. Chamber 1 has been pre-coated with immobilized probes A, B, and C, which are designed to complementarily bind via thermo-chemical interaction, to any DNA fragments featuring, respectively, target DNA sequences A, B, and C. When the sample comprising unique DNA fragment types 1-18 are first loaded into chamber 1, all DNA fragments featuring target DNA sequence A bind to immobilized probe A, all DNA fragments featuring target DNA sequence B bind to immobilized probe B, and all DNA fragments featuring target DNA sequence C bind to immobilized probe C. Thus, any DNA fragments with target DNA sequences A, B, and C are essentially extracted from the sample and retained in chamber 1. The rest of the sample is flowed to chamber 2, thus physically separating those three types of DNA fragments from the rest of the sample.

Each of the remaining separation and detection chambers have also been pre-coated with three different immobilized probes, for a total of eighteen different immobilized probes in the biochip to capture the eighteen different DNA fragment types featuring eighteen target DNA sequences. In a similar manner, the sample is sequentially flowed through the separation and detection chambers, one chamber at a time, resulting in the extraction and physical separation of DNA fragments into the six separation and detection chambers according to the presence of target DNA sequences. The immobilized probes are designed or chosen for each separation and detection chamber in order to bind three different types of DNA fragments, which are labeled with three different colors.

Following space separation, the three-color fluorescence detection system is used to detect the fluorescence emissions from each biochip separation and detection chamber, which contains paired immobilized probes and DNA fragments that emit three fluorescent colors. Because the unique types of DNA fragments have been physically separated into six separation and detection chambers according to target DNA sequence, up to six types of DNA fragments still may be labeled with an identical fluorescent color and be detected by a three-color fluorescence detection system. In total, all eighteen different target DNA sequences may be detected and analyzed.

### Example 3

In a third exemplary embodiment, a biochip with six separation and detection chambers was used with a single-color fluorescence detection system to simultaneously detect as many as six target DNA sequences. A multiplex PCR or isothermal amplification process was used to generate a sample comprising six unique types of DNA fragments, which were of similar or different size. All six types of DNA fragments were labeled with an identical fluorescent color. The sample (e.g., 20 μL) might be re-suspended in a buffer (e.g., 30 μL) in order to begin with a higher sample volume (e.g., 50 μL).

Instead of sequentially flowing the sample through the biochip separation and detection chambers, one chamber at a time, the sample was simultaneously loaded into all six separation and detection chambers (e.g., about 8 μL volume per chamber). Each of the six separation and detection chambers had a probe corresponding to one DNA fragment for complementary binding. Hence in chamber 1, DNA fragments with DNA sequence A bound to probe A, while the other types of DNA fragments present in the sample in this chamber had no complementary binding to probe A. Similarly, in chamber 2, DNA fragments with DNA sequence B bound to probe B, while the other types of DNA fragments present in the sample in this chamber had no complementary binding to probe B. The process was similar for the remaining four separation and detection chambers.

One advantage of this approach is that, a probe in a separation and detection chamber does not need to be immobilized to the surface of the chamber. As illustrated above, this is because the amplified DNA fragments does not flow sequentially through all of the separation and detection chambers and a wash buffer is optional. Instead, all of the separation and detection chambers were filled simultaneously. However, a quencher oligonucleotide might be required to quench the fluorescence emission of any DNA fragment that did not bind to a probe. The absence of flow suggested that complementarily-bound DNA fragment-probe pairs (along with other unbound macromolecules present in the 8 μL sample) remained within the separation and detection chamber for subsequent fluorescent detection.

In this example, the fluorescence emission from a separation and detection chamber indicated the presence (or absence in the case of a fluorescence reduction method) of successful DNA fragment-probe complementary binding and the concentration thereof. All six types of DNA fragments was detected in the six independent separation and detection chambers using a single-color fluorescence detection system.

### Example 4

To demonstrate the common quencher method for detecting multiplex PCR, we performed the following amplification and detection experiment on a biochip (NanoMDx, MA). For this demonstration, a biochip described in example 3, but with 2 fluorescent colors, was utilized.

Ultramer/DNA of 12 respiratory pathogens (purchased from IDT Technologies, CA) at 100000 copies each were co-amplified in a single PCR reaction of c.a. 25 μL. PCR chemistry contained final primers concentration of 0.2 μM each for the 12 primers, and the Qiagen multiplex PCR Kit (cat#206152, Qiagen, CA) and HotStar polymerase (1U per reaction) were utilized to constitute the reaction. PCR thermal cycling conditions were, 96° C. for 10 min for initial denaturation followed by 40 cycles of 96° C. for 1 min, 60° C. for 1 min, 72° C. for 1 min, and final extension at 72° C. for 5 min. Upon completion of PCR amplification, ˜70 μL of water was used as buffer to reconstitute the PCR amplicons to a total volume of about 90 μL. This volume was then simultaneously input into six detection chambers. Each of the six detection chambers contained probe and quencher for only 2 targets, of which one target was labeled with FAM dye and the other with Cy5 dye. Hence with 2 distinct fluorescence probes/quencher in each of the 6 chambers a total of 12 targets is detected from the multiplex PCR amplification. The primers, probes, and quencher sequences are listed in the table below.

In each of the six chambers, the probe and the common-quencher oligonucleotide containing BHQ quencher was at equimolar concentration of 0.2 μM contained in ˜5 μL solution. With a detection volume capacity of ˜20 μL, an c.a. of 15 μL of suspended PCR amplicon volume, and 5 μL of probe/common-quencher is present in each detection chamber.

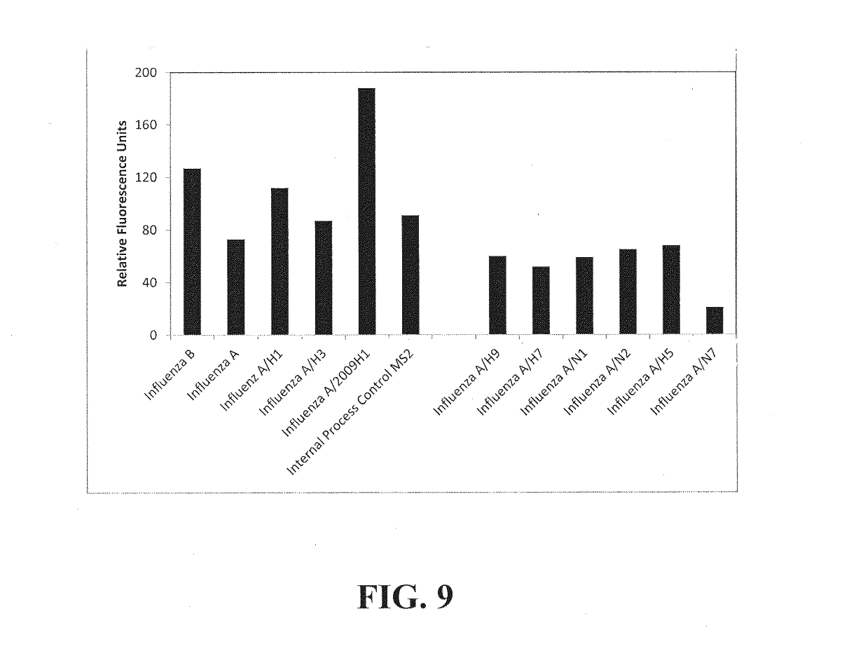

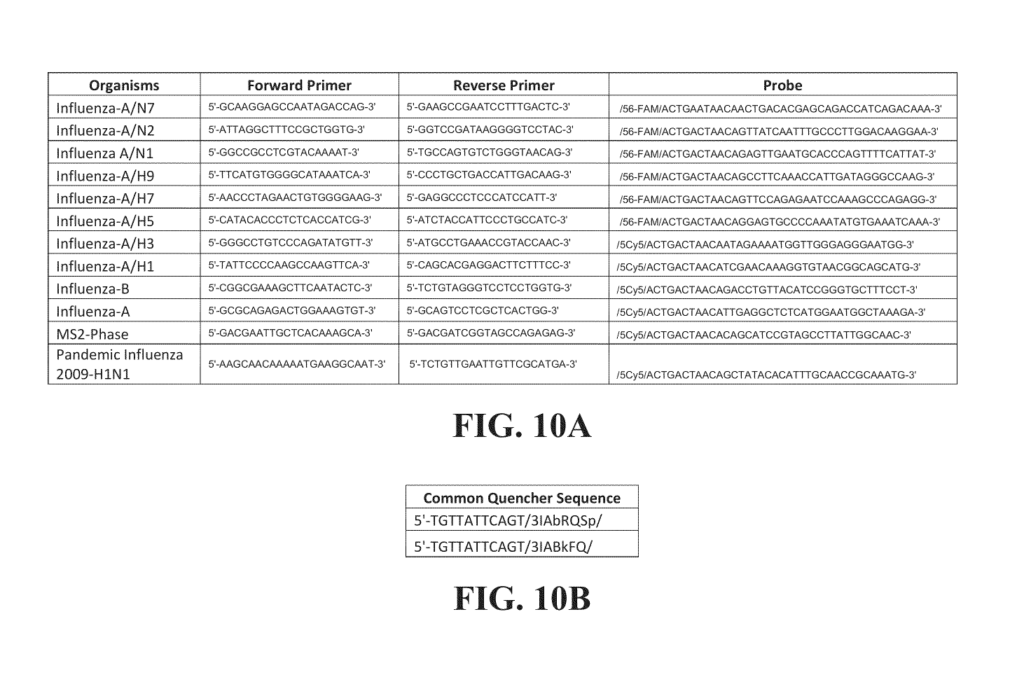

Once the PCR amplicon was loaded into all 6 detection chambers simultaneously, the temperature was ramped from ˜25° C. (room temperature) to 94° C. at the rate of 1.5° C.-2.5° C. per second. After the temperature reached 94° C., a ramp down was initiated from 94° C. to 25° C. at a ramp down rate of 2-4° C. per second. This ramp up and down allows for the different denaturing and annealing interactions between the PCR amplified amplicons, probes, and primers, as described earlier. Finally, the fluorescence of FAM and Cy5 in each of the 6 chambers was measured on a fluorescence reader (FLX800T, BioTek Instruments, VT), data shown below. Presence of FAM and Cy5 fluorescence in each chamber indicates the successful binding of a PCR amplicon to the corresponding detection probe present in that chamber. As shown in FIG. 9, an increase in probe fluorescence, represented in relative fluorescence units (RFU), is noted for each of the 12 targets. FIG. 10A lists forward primers, reverse primers, and probe sequences for the twelve targets. FIG. 10B lists two exemplary common quenchers with their sequences and dye quenching moieties used in the experiment. The random sequence for both examples is TGTTATTCAGT, and the dye quenching moieties are 31AbRQSp and 31ABkFQ.

While the above demonstrated the detection of 12 targets in 6 chambers with 2 color detection in each chamber, it is possible to increase the number of detection targets, for example to 4 in each chamber, to achieve a distinct fluorescence detection of 24 targets in 6 chambers. Increasing the number of detection chambers beyond 6 also provides increased target detection ability. The above experiment validated one of our unique simultaneous/parallel detection methods and the unique common-quencher method. Probes that are bound to the DNA strand emits fluorescence. And the remaining probes are bound to the common quencher oligonucleotides, and therefore these remaining probes do not emit fluorescence.

A person of ordinary skill will understand that these different approaches may be combined in different embodiments. For instance, the three-color fluorescence detection system approach in Example 2 can also be applied to the method of Example 3 to detect up to three different target DNA sequences in each separation and detection chamber for a total of up to eighteen different target DNA sequences across a biochip with six separation and detection chambers.

Also, as illustrated above, the scope of the present invention is not limited to a biochip having multiple chambers, but the detection method can use any other multiple enclosures (e.g., vials or chambers).

The following lists provide examples of common quencher sequence listings.

LIST 1

>1|random sequence|A: 0.3|C: 0.2|G: 0.2|T: 0.23|length: 12 bp
aacgtgactttt
>2|random sequence|A: 0.3|C: 0.2|G: 0.2|T: 0.23|length: 9 bp
attgtatct
>3|random sequence|A: 0.3|C: 0.2|G: 0.2|T: 0.23|length: 17 bp
gtcctttattgcaagaa
>4|random sequence|A: 0.3|C: 0.2|G: 0.2|T: 0.23|length: 10 bp
actggaattc
>5|random sequence|A: 0.3|C: 0.2|G: 0.2|T: 0.23|length: 14 bp
ttagttcatagcat
>6|random sequence|A: 0.3|C: 0.2|G: 0.2|T: 0.23|length: 13 bp
atgtattattccg
>7|random sequence|A: 0.3|C: 0.2|G: 0.2|T: 0.23|length: 10 bp
attatggcac
>8|random sequence|A: 0.3|C: 0.2|G: 0.2|T: 0.23|length: 8 bp
catagttt
>9|random sequence|A: 0.3|C: 0.2|G: 0.2|T: 0.23|length: 14 bp
atttcctgtaatag
>10|random sequence|A: 0.3|C: 0.2|G: 0.2|T: 0.23|length: 12 bp
gatgtctcatta
>11|random sequence|A: 0.3|C: 0.2|G: 0.2|T: 0.23|length: 14 bp
ctacgtttttagaa
>12|random sequence|A: 0.3|C: 0.2|G: 0.2|T: 0.23|length: 12 bp
taagcgtttcat
>13|random sequence|A: 0.3|C: 0.2|G: 0.2|T: 0.23|length: 14 bp
gaccgtattaattt
>14|random sequence|A: 0.3|C: 0.2|G: 0.2|T: 0.23|length: 13 bp
attagtttctgac
>15|random sequence|A: 0.3|C: 0.2|G: 0.2|T: 0.23|length: 18 bp
gtatccattatgagtatc
>16|random sequence|A: 0.3|C: 0.2|G: 0.2|T: 0.23|length: 8 bp
ttttcgaa
>17|random sequence|A: 0.3|C: 0.2|G: 0.2|T: 0.23|length: 16 bp
ttctacgatgctaatg
>18|random sequence|A: 0.3|C: 0.2|G: 0.2|T: 0.23|length: 13 bp
catgatattctgt
>19|random sequence|A: 0.3|C: 0.2|G: 0.2|T: 0.23|length: 19 bp
attaatgtccaagtttgct
>20|random sequence|A: 0.3|C: 0.2|G: 0.2|T: 0.23|length: 17 bp
ttgcattagttcaaacg
>21|random sequence|A: 0.3|C: 0.2|G: 0.2|T: 0.23|length: 9 bp
tacttttag
>22|random sequence|A: 0.3|C: 0.2|G: 0.2|T: 0.23|length: 16 bp
gttgctaaagcattct
>23|random sequence|A: 0.3|C: 0.2|G: 0.2|T: 0.23|length: 16 bp
gagctatgtttcaatc
>24|random sequence|A: 0.3|C: 0.2|G: 0.2|T: 0.23|length: 10 bp
atccgtagta
>25|random sequence|A: 0.3|C: 0.2|G: 0.2|T: 0.23|length: 11 bp
attgacgtcat
>26|random sequence|A: 0.3|C: 0.2|G: 0.2|T: 0.23|length: 12 bp
gctgttcaatat
>27|random sequence|A: 0.3|C: 0.2|G: 0.2|T: 0.23|length: 14 bp
tacggttacattta
>28|random sequence|A: 0.3|C: 0.2|G: 0.2|T: 0.23|length: 15 bp
tattacgctagagct
>29|random sequence|A: 0.3|C: 0.2|G: 0.2|T: 0.23|length: 15 bp
gtatatccgctgtaa
>30|random sequence|A: 0.3|C: 0.2|G: 0.2|T: 0.23|length: 17 bp
cgtctaattaggacatt
>31|random sequence|A: 0.3|C: 0.2|G: 0.2|T: 0.23|length: 20 bp
tcatatattactaggacggc
>32|random sequence|A: 0.3|C: 0.2|G: 0.2|T: 0.23|length: 9 bp
tatactttg
>33|random sequence|A: 0.3|C: 0.2|G: 0.2|T: 0.23|length: 15 bp
tatgtgactcaatcg
>34|random sequence|A: 0.3|C: 0.2|G: 0.2|T: 0.23|length: 20 bp
atcaagcttcgtagtgctaa
>35|random sequence|A: 0.3|C: 0.2|G: 0.2|T: 0.23|length: 15 bp
tacattagccgtagt
>36|random sequence|A: 0.3|C: 0.2|G: 0.2|T: 0.23|length: 11 bp
tatccagtagt
>37|random sequence|A: 0.3|C: 0.2|G: 0.2|T: 0.23|length: 11 bp
ttatggcctaa
>38|random sequence|A: 0.3|C: 0.2|G: 0.2|T: 0.23|length: 9 bp
gattcttta
>39|random sequence|A: 0.3|C: 0.2|G: 0.2|T: 0.23|length: 16 bp
aatcgtatccagtgtt
>40|random sequence|A: 0.3|C: 0.2|G: 0.2|T: 0.23|length: 20 bp
tggtaaaactagactccgtt
>41|random sequence|A: 0.3|C: 0.2|G: 0.2|T: 0.23|length: 16 bp
ttagcgtattatcagc
>42|random sequence|A: 0.3|C: 0.2|G: 0.2|T: 0.23|length: 20 bp
gttgactaatgagcactatc
>43|random sequence|A: 0.3|C: 0.2|G: 0.2|T: 0.23|length: 10 bp
cttagatagc
>44|random sequence|A: 0.3|C: 0.2|G: 0.2|T: 0.23|length: 14 bp
aattacagtttctg
>45|random sequence|A: 0.3|C: 0.2|G: 0.2|T: 0.23|length: 11 bp
agcatatttgc
>46|random sequence|A: 0.3|C: 0.2|G: 0.2|T: 0.23|length: 14 bp
atgtcactatgtta
>47|random sequence|A: 0.3|C: 0.2|G: 0.2|T: 0.23|length: 13 bp
tcacttatgtgta
>48|random sequence|A: 0.3|C: 0.2|G: 0.2|T: 0.23|length: 18 bp
ccaagtttttcagagtta
>49|random sequence|A: 0.3|C: 0.2|G: 0.2|T: 0.23|length: 9 bp
atcagtttt
>50|random sequence|A: 0.3|C: 0.2|G: 0.2|T: 0.23|length: 18 bp
tgctttaatatccaggat
>51|random sequence|A: 0.3|C: 0.2|G: 0.2|T: 0.23|length: 19 bp
tttttgacaataatgcgtc
>52|random sequence|A: 0.3|C: 0.2|G: 0.2|T: 0.23|length: 12 bp
taatatgcttcg
>53|random sequence|A: 0.3|C: 0.2|G: 0.2|T: 0.23|length: 18 bp
aatatgtaccgttgtcat
>54|random sequence|A: 0.3|C: 0.2|G: 0.2|T: 0.23|length: 9 bp
tgtattcta
>55|random sequence|A: 0.3|C: 0.2|G: 0.2|T: 0.23|length: 14 bp
acatacttaggttt
>56|random sequence|A: 0.3|C: 0.2|G: 0.2|T: 0.23|length: 19 bp
tagccttcagtaagtttat
>57|random sequence|A: 0.3|C: 0.2|G: 0.2|T: 0.23|length: 16 bp
agatctagtgcttcat
>58|random sequence|A: 0.3|C: 0.2|G: 0.2|T: 0.23|length: 12 bp
atttgccttgaa
>59|random sequence|A: 0.3|C: 0.2|G: 0.2|T: 0.23|length: 13 bp
tgtctgactttaa
>60|random sequence|A: 0.3|C: 0.2|G: 0.2|T: 0.23|length: 10 bp
gatatacctg
>61|random sequence|A: 0.3|C: 0.2|G: 0.2|T: 0.23|length: 18 bp
actgaatgattattcctg
>62|random sequence|A: 0.3|C: 0.2|G: 0.2|T: 0.23|length: 19 bp
gtcggtatttaatcactat
>63|random sequence|A: 0.3|C: 0.2|G: 0.2|T: 0.23|length: 16 bp
attcgatcgtcatgat
>64|random sequence|A: 0.3|C: 0.2|G: 0.2|T: 0.23|length: 9 bp
ttcgtaatt
>65|random sequence|A: 0.3|C: 0.2|G: 0.2|T: 0.23|length: 13 bp
tcgtttaatgcat
>66|random sequence|A: 0.3|C: 0.2|G: 0.2|T: 0.23|length: 12 bp
tcagatcttatg
>67|random sequence|A: 0.3|C: 0.2|G: 0.2|T: 0.23|length: 19 bp
agttcttaaccgtatattg
>68|random sequence|A: 0.3|C: 0.2|G: 0.2|T: 0.23|length: 15 bp
aatctcctaattggg
>69|random sequence|A: 0.3|C: 0.2|G: 0.2|T: 0.23|length: 14 bp
tctaaggcatttat
>70|random sequence|A: 0.3|C: 0.2|G: 0.2|T: 0.23|length: 13 bp
atctttagtgact
>71|random sequence|A: 0.3|C: 0.2|G: 0.2|T: 0.23|length: 11 bp
aattgcgatct
>72|random sequence|A: 0.3|C: 0.2|G: 0.2|T: 0.23|length: 12 bp
cgatcagtttat
>73|random sequence|A: 0.3|C: 0.2|G: 0.2|T: 0.23|length: 20 bp
gcggtcaatatgctacatta
>74|random sequence|A: 0.3|C: 0.2|G: 0.2|T: 0.23|length: 15 bp
cgctatttagaatgc
>75|random sequence|A: 0.3|C: 0.2|G: 0.2|T: 0.23|length: 16 bp
gtctgatatacagtct
>76|random sequence|A: 0.3|C: 0.2|G: 0.2|T: 0.23|length: 9 bp
ttacgattt
>77|random sequence|A: 0.3|C: 0.2|G: 0.2|T: 0.23|length: 20 bp
agagtatcgtcgaattacct
>78|random sequence|A: 0.3|C: 0.2|G: 0.2|T: 0.23|length: 8 bp
catgttat
>79|random sequence|A: 0.3|C: 0.2|G: 0.2|T: 0.23|length: 12 bp
acgtatttactg
>80|random sequence|A: 0.3|C: 0.2|G: 0.2|T: 0.23|length: 15 bp
ttcgtacctaagtag
>81|random sequence|A: 0.3|C: 0.2|G: 0.2|T: 0.23|length: 12 bp
cagtgcttaatt
>82|random sequence|A: 0.3|C: 0.2|G: 0.2|T: 0.23|length: 20 bp
cttcacaattgtactgggaa
>83|random sequence|A: 0.3|C: 0.2|G: 0.2|T: 0.23|length: 16 bp
tattactgggcactat
>84|random sequence|A: 0.3|C: 0.2|G: 0.2|T: 0.23|length: 20 bp
acatcttcggcaatttgaag
>85|random sequence|A: 0.3|C: 0.2|G: 0.2|T: 0.23|length: 19 bp
agatttccagtctgtatta
>86|random sequence|A: 0.3|C: 0.2|G: 0.2|T: 0.23|length: 16 bp
gttacctaatgctagt
>87|random sequence|A: 0.3|C: 0.2|G: 0.2|T: 0.23|length: 12 bp
catttaatggtc
>88|random sequence|A: 0.3|C: 0.2|G: 0.2|T: 0.23|length: 8 bp
tactttag
>89|random sequence|A: 0.3|C: 0.2|G: 0.2|T: 0.23|length: 10 bp
cgattcaagt
>90|random sequence|A: 0.3|C: 0.2|G: 0.2|T: 0.23|length: 10 bp
tatacagtgc
>91|random sequence|A: 0.3|C: 0.2|G: 0.2|T: 0.23|length: 15 bp
tccaaggttaatgtc
>92|random sequence|A: 0.3|C: 0.2|G: 0.2|T: 0.23|length: 8 bp
actttagt
>93|random sequence|A: 0.3|C: 0.2|G: 0.2|T: 0.23|length: 13 bp
tatctcgtaattg
>94|random sequence|A: 0.3|C: 0.2|G: 0.2|T: 0.23|length: 20 bp
atatcatacccgagtagttg
>95|random sequence|A: 0.3|C: 0.2|G: 0.2|T: 0.23|length: 10 bp
aggtctaact
>96|random sequence|A: 0.3|C: 0.2|G: 0.2|T: 0.23|length: 14 bp
tatggttactacta
>97|random sequence|A: 0.3|C: 0.2|G: 0.2|T: 0.23|length: 11 bp
gaacttgctta
>98|random sequence|A: 0.3|C: 0.2|G: 0.2|T: 0.23|length: 19 bp
cagtggtaattccatattt
>99|random sequence|A: 0.3|C: 0.2|G: 0.2|T: 0.23|length: 8 bp
tacagttt
>100|random sequence|A: 0.3|C: 0.2|G: 0.2|T: 0.23|length: 18 bp
gagttaatttcacattcg

LIST 2

>1|random sequence|A: 0.25|C: 0.25|G: 0.25|T: 0.25|length: 16 bp
gacctaacttcagggt
>2|random sequence|A: 0.25|C: 0.25|G: 0.25|T: 0.25|length: 19 bp
ctaggcagttcatgcttat
>3|random sequence|A: 0.25|C: 0.25|G: 0.25|T: 0.25|length: 15 bp
acctgctattaggtt
>4|random sequence|A: 0.25|C: 0.25|G: 0.25|T: 0.25|length: 15 bp
ctcttcatggatatg
>5|random sequence|A: 0.25|C: 0.25|G: 0.25|T: 0.25|length: 14 bp
cttacttgtggaac
>6|random sequence|A: 0.25|C: 0.25|G: 0.25|T: 0.25|length: 11 bp
tttgacacttg
>7|random sequence|A: 0.25|C: 0.25|G: 0.25|T: 0.25|length: 20 bp
gccgagtcagaattgttcac
>8|random sequence|A: 0.25|C: 0.25|G: 0.25|T: 0.25|length: 12 bp
tctaatggcagc
>9|random sequence|A: 0.25|C: 0.25|G: 0.25|T: 0.25|length: 12 bp
cgactatgtcag
>10|random sequence|A: 0.25|C: 0.25|G: 0.25|T: 0.25|length: 12 bp
tcgacttcagga
>11|random sequence|A: 0.25|C: 0.25|G: 0.25|T: 0.25|length: 20 bp
ctttgtgcaaagcgagtcac
>12|random sequence|A: 0.25|C: 0.25|G: 0.25|T: 0.25|length: 15 bp
cgtatgtatcattgc
>13|random sequence|A: 0.25|C: 0.25|G: 0.25|T: 0.25|length: 9 bp
ctcggaatt
>14|random sequence|A: 0.25|C: 0.25|G: 0.25|T: 0.25|length: 10 bp
ccattttgag
>15|random sequence|A: 0.25|C: 0.25|G: 0.25|T: 0.25|length: 11 bp
gcttctagtta
>16|random sequence|A: 0.25|C: 0.25|G: 0.25|T: 0.25|length: 20 bp
aattacgccaaccttgggtg
>17|random sequence|A: 0.25|C: 0.25|G: 0.25|T: 0.25|length: 16 bp
acctacgtttaggcga
>18|random sequence|A: 0.25|C: 0.25|G: 0.25|T: 0.25|length: 18 bp
gcggaattttcactactg
>19|random sequence|A: 0.25|C: 0.25|G: 0.25|T: 0.25|length: 17 bp
tggactaatcacggttc
>20|random sequence|A: 0.25|C: 0.25|G: 0.25|T: 0.25|length: 8 bp
cgtcaatg
>21|random sequence|A: 0.25|C: 0.25|G: 0.25|T: 0.25|length: 16 bp
atctgtgcaggtcaac
>22|random sequence|A: 0.25|C: 0.25|G: 0.25|T: 0.25|length: 19 bp
cagtactctagggactttt
>23|random sequence|A: 0.25|C: 0.25|G: 0.25|T: 0.25|length: 8 bp
ttaacgcg
>24|random sequence|A: 0.25|C: 0.25|G: 0.25|T: 0.25|length: 13 bp
ttgtcagctagca
>25|random sequence|A: 0.25|C: 0.25|G: 0.25|T: 0.25|length: 19 bp
cttctggagcgtctaatat
>26|random sequence|A: 0.25|C: 0.25|G: 0.25|T: 0.25|length: 10 bp
gctaagttct
>27|random sequence|A: 0.25|C: 0.25|G: 0.25|T: 0.25|length: 8 bp
tcgcagat
>28|random sequence|A: 0.25|C: 0.25|G: 0.25|T: 0.25|length: 9 bp
gagtactct
>29|random sequence|A: 0.25|C: 0.25|G: 0.25|T: 0.25|length: 8 bp
agtagcct
>30|random sequence|A: 0.25|C: 0.25|G: 0.25|T: 0.25|length: 20 bp
tacactggttaggcatcacg
>31|random sequence|A: 0.25|C: 0.25|G: 0.25|T: 0.25|length: 19 bp
ccagtgcatacgtttttga
>32|random sequence|A: 0.25|C: 0.25|G: 0.25|T: 0.25|length: 15 bp
ctttctcgattagga
>33|random sequence|A: 0.25|C: 0.25|G: 0.25|T: 0.25|length: 14 bp
actgactttgcatg
>34|random sequence|A: 0.25|C: 0.25|G: 0.25|T: 0.25|length: 17 bp
cggccaattcgaatttg
>35|random sequence|A: 0.25|C: 0.25|G: 0.25|T: 0.25|length: 12 bp
tcgcagatacgt
>36|random sequence|A: 0.25|C: 0.25|G: 0.25|T: 0.25|length: 12 bp
gaacctgtagtc
>37|random sequence|A: 0.25|C: 0.25|G: 0.25|T: 0.25|length: 19 bp
attagtatggtccctatgc
>38|random sequence|A: 0.25|C: 0.25|G: 0.25|T: 0.25|length: 11 bp
gcattgttact
>39|random sequence|A: 0.25|C: 0.25|G: 0.25|T: 0.25|length: 17 bp
atggatcgctcttacag
>40|random sequence|A: 0.25|C: 0.25|G: 0.25|T: 0.25|length: 19 bp
gcatgtgccgtttctaata
>41|random sequence|A: 0.25|C: 0.25|G: 0.25|T: 0.25|length: 17 bp
aacgcatcgtatcggtt
>42|random sequence|A: 0.25|C: 0.25|G: 0.25|T: 0.25|length: 14 bp
ttgacgtccatgat
>43|random sequence|A: 0.25|C: 0.25|G: 0.25|T: 0.25|length: 12 bp
ccagacttagtg
>44|random sequence|A: 0.25|C: 0.25|G: 0.25|T: 0.25|length: 20 bp
atctgaagtggagcactctc
>45|random sequence|A: 0.25|C: 0.25|G: 0.25|T: 0.25|length: 13 bp
gatcccgatatgt
>46|random sequence|A: 0.25|C: 0.25|G: 0.25|T: 0.25|length: 19 bp
tactgtaggatgctctcta
>47|random sequence|A: 0.25|C: 0.25|G: 0.25|T: 0.25|length: 16 bp
caactttcgcaaggtg
>48|random sequence|A: 0.25|C: 0.25|G: 0.25|T: 0.25|length: 13 bp
gtctgcataatgc
>49|random sequence|A: 0.25|C: 0.25|G: 0.25|T: 0.25|length: 12 bp
caatggtccatg
>50|random sequence|A: 0.25|C: 0.25|G: 0.25|T: 0.25|length: 11 bp
tgcaatttcgt
>51|random sequence|A: 0.25|C: 0.25|G: 0.25|T: 0.25|length: 15 bp
tatcgctattgctga
>52|random sequence|A: 0.25|C: 0.25|G: 0.25|T: 0.25|length: 16 bp
tatgtcgcgagccaat
>53|random sequence|A: 0.25|C: 0.25|G: 0.25|T: 0.25|length: 20 bp
ttgacacgtgaatgccactg
>54|random sequence|A: 0.25|C: 0.25|G: 0.25|T: 0.25|length: 15 bp
gagcttcttattgac
>55|random sequence|A: 0.25|C: 0.25|G: 0.25|T: 0.25|length: 16 bp
acgacttgatgtgcca
>56|random sequence|A: 0.25|C: 0.25|G: 0.25|T: 0.25|length: 9 bp
ctatcgtga
>57|random sequence|A: 0.25|C: 0.25|G: 0.25|T: 0.25|length: 17 bp
tcacgattagcttcagg
>58|random sequence|A: 0.25|C: 0.25|G: 0.25|T: 0.25|length: 16 bp
tggcgatagactccta
>59|random sequence|A: 0.25|C: 0.25|G: 0.25|T: 0.25|length: 15 bp
aattgcgtcttctag
>60|random sequence|A: 0.25|C: 0.25|G: 0.25|T: 0.25|length: 14 bp
gttagtatccgtca
>61|random sequence|A: 0.25|C: 0.25|G: 0.25|T: 0.25|length: 18 bp
tgatcgtaggtatctacc
>62|random sequence|A: 0.25|C: 0.25|G: 0.25|T: 0.25|length: 10 bp
agtcttgtca
>63|random sequence|A: 0.25|C: 0.25|G: 0.25|T: 0.25|length: 11 bp
ttcgtatgatc
>64|random sequence|A: 0.25|C: 0.25|G: 0.25|T: 0.25|length: 13 bp
gcaggatctttca
>65|random sequence|A: 0.25|C: 0.25|G: 0.25|T: 0.25|length: 8 bp
tcgacgta
>66|random sequence|A: 0.25|C: 0.25|G: 0.25|T: 0.25|length: 16 bp
gatggagcattcccat
>67|random sequence|A: 0.25|C: 0.25|G: 0.25|T: 0.25|length: 18 bp
tgatatacagtctgcgtc
>68|random sequence|A: 0.25|C: 0.25|G: 0.25|T: 0.25|length: 9 bp
agtcacgtt
>69|random sequence|A: 0.25|C: 0.25|G: 0.25|T: 0.25|length: 19 bp
agccgatctaggctatttt
>70|random sequence|A: 0.25|C: 0.25|G: 0.25|T: 0.25|length: 14 bp
tagatctcactggt
>71|random sequence|A: 0.25|C: 0.25|G: 0.25|T: 0.25|length: 9 bp
tgccagatt
>72|random sequence|A: 0.25|C: 0.25|G: 0.25|T: 0.25|length: 10 bp
cgtctgatat
>73|random sequence|A: 0.25|C: 0.25|G: 0.25|T: 0.25|length: 13 bp
gcttggacactta
>74|random sequence|A: 0.25|C: 0.25|G: 0.25|T: 0.25|length: 17 bp
ccaggtagttcctagat
>75|random sequence|A: 0.25|C: 0.25|G: 0.25|T: 0.25|length: 10 bp
tcgtgacatt
>76|random sequence|A: 0.25|C: 0.25|G: 0.25|T: 0.25|length: 19 bp
ctctaccgatatgtgattg
>77|random sequence|A: 0.25|C: 0.25|G: 0.25|T: 0.25|length: 9 bp
tagtcatgc
>78|random sequence|A: 0.25|C: 0.25|G: 0.25|T: 0.25|length: 13 bp
tagctagactcgt
>79|random sequence|A: 0.25|C: 0.25|G: 0.25|T: 0.25|length: 18 bp
ctttgaatgtagagctcc
>80|random sequence|A: 0.25|C: 0.25|G: 0.25|T: 0.25|length: 18 bp
taggctgatttcagtacc
>81|random sequence|A: 0.25|C: 0.25|G: 0.25|T: 0.25|length: 13 bp
attaagtctggcc
>82|random sequence|A: 0.25|C: 0.25|G: 0.25|T: 0.25|length: 9 bp
tacgttcag
>83|random sequence|A: 0.25|C: 0.25|G: 0.25|T: 0.25|length: 18 bp
gcgtcaatctgttcaatg
>84|random sequence|A: 0.25|C: 0.25|G: 0.25|T: 0.25|length: 16 bp
acgtagaatgtctcgc
>85|random sequence|A: 0.25|C: 0.25|G: 0.25|T: 0.25|length: 12 bp
gaagtgcttcca
>86|random sequence|A: 0.25|C: 0.25|G: 0.25|T: 0.25|length: 11 bp
ttgagttctca
>87|random sequence|A: 0.25|C: 0.25|G: 0.25|T: 0.25|length: 8 bp
tgcagtac
>88|random sequence|A: 0.25|C: 0.25|G: 0.25|T: 0.25|length: 16 bp
ctctactgagcgatag
>89|random sequence|A: 0.25|C: 0.25|G: 0.25|T: 0.25|length: 13 bp
ttagcgcagtcta
>90|random sequence|A: 0.25|C: 0.25|G: 0.25|T: 0.25|length: 14 bp
gcttgacctttaag
>91|random sequence|A: 0.25|C: 0.25|G: 0.25|T: 0.25|length: 17 bp
tgctatgaaggatccct
>92|random sequence|A: 0.25|C: 0.25|G: 0.25|T: 0.25|length: 20 bp
ggcgccctatagagtactat
>93|random sequence|A: 0.25|C: 0.25|G: 0.25|T: 0.25|length: 15 bp
aatatccgttcgttg
>94|random sequence|A: 0.25|C: 0.25|G: 0.25|T: 0.25|length: 20 bp
gctcgtagtcgacaactgta
>95|random sequence|A: 0.25|C: 0.25|G: 0.25|T: 0.25|length: 19 bp
ggcttagactttgtaatcc
>96|random sequence|A: 0.25|C: 0.25|G: 0.25|T: 0.25|length: 18 bp
taattggatccaccgttg
>97|random sequence|A: 0.25|C: 0.25|G: 0.25|T: 0.25|length: 9 bp
gactttcag
>98|random sequence|A: 0.25|C: 0.25|G: 0.25|T: 0.25|length: 13 bp
catgtcctgtaga
>99|random sequence|A: 0.25|C: 0.25|G: 0.25|T: 0.25|length: 8 bp
tgacagct
>100|random sequence|A: 0.25|C: 0.25|G: 0.25|T: 0.25|length: 15 bp
caatgactttgttgc

LIST 3

>1|random sequence|A: 0.35|C: 0.15|G: 0.15|T: 0.35|length: 20 bp
ctatgataatcaatgcattg
>2|random sequence|A: 0.35|C: 0.15|G: 0.15|T: 0.35|length: 8 bp
ttcatgat
>3|random sequence|A: 0.35|C: 0.15|G: 0.15|T: 0.35|length: 18 bp
ttgagttaatcactatat
>4|random sequence|A: 0.35|C: 0.15|G: 0.15|T: 0.35|length: 14 bp
gttataaattcgtc
>5|random sequence|A: 0.35|C: 0.15|G: 0.15|T: 0.35|length: 12 bp
ataatatttgct
>6|random sequence|A: 0.35|C: 0.15|G: 0.15|T: 0.35|length: 17 bp
aaatttttgctttgaca
>7|random sequence|A: 0.35|C: 0.15|G: 0.15|T: 0.35|length: 8 bp
acttatgt
>8|random sequence|A: 0.35|C: 0.15|G: 0.15|T: 0.35|length: 19 bp
cttttataaatcgtagtat
>9|random sequence|A: 0.35|C: 0.15|G: 0.15|T: 0.35|length: 20 bp
aagtcatcattgtacatagt
>10|random sequence|A: 0.35|C: 0.15|G: 0.15|T: 0.35|length: 16 bp
actagttaatttgatc
>11|random sequence|A: 0.35|C: 0.15|G: 0.15|T: 0.35|length: 10 bp
tactttagat
>12|random sequence|A: 0.35|C: 0.15|G: 0.15|T: 0.35|length: 19 bp
aattcatatgcttagattt
>13|random sequence|A: 0.35|C: 0.15|G: 0.15|T: 0.35|length: 12 bp
tagttacatatt
>14|random sequence|A: 0.35|C: 0.15|G: 0.15|T: 0.35|length: 11 bp
aattttgttac
>15|random sequence|A: 0.35|C: 0.15|G: 0.15|T: 0.35|length: 11 bp
tatatttgcat
>16|random sequence|A: 0.35|C: 0.15|G: 0.15|T: 0.35|length: 14 bp
gattctgatatatc
>17|random sequence|A: 0.35|C: 0.15|G: 0.15|T: 0.35|length: 11 bp
gatcattttat
>18|random sequence|A: 0.35|C: 0.15|G: 0.15|T: 0.35|length: 18 bp
ttttataatcaggacatt
>19|random sequence|A: 0.35|C: 0.15|G: 0.15|T: 0.35|length: 16 bp
ttcaatatctagtatg
>20|random sequence|A: 0.35|C: 0.15|G: 0.15|T: 0.35|length: 12 bp
tatatttgatac
>21|random sequence|A: 0.35|C: 0.15|G: 0.15|T: 0.35|length: 9 bp
catatgtta
>22|random sequence|A: 0.35|C: 0.15|G: 0.15|T: 0.35|length: 13 bp
ttcttgataatat
>23|random sequence|A: 0.35|C: 0.15|G: 0.15|T: 0.35|length: 16 bp
aagttattcttatcag
>24|random sequence|A: 0.35|C: 0.15|G: 0.15|T: 0.35|length: 19 bp
attacgtactatttgatta
>25|random sequence|A: 0.35|C: 0.15|G: 0.15|T: 0.35|length: 15 bp
ctctgattagaaatt
>26|random sequence|A: 0.35|C: 0.15|G: 0.15|T: 0.35|length: 20 bp
tgccatttaataggatctaa
>27|random sequence|A: 0.35|C: 0.15|G: 0.15|T: 0.35|length: 14 bp
attcttttacagag
>28|random sequence|A: 0.35|C: 0.15|G: 0.15|T: 0.35|length: 16 bp
aagtatattttccatg
>29|random sequence|A: 0.35|C: 0.15|G: 0.15|T: 0.35|length: 11 bp
atctattagtt
>30|random sequence|A: 0.35|C: 0.15|G: 0.15|T: 0.35|length: 19 bp
ctttgataatgatttatac
>31|random sequence|A: 0.35|C: 0.15|G: 0.15|T: 0.35|length: 13 bp
tagttaattctta
>32|random sequence|A: 0.35|C: 0.15|G: 0.15|T: 0.35|length: 14 bp
ataatattcgtcgt
>33|random sequence|A: 0.35|C: 0.15|G: 0.15|T: 0.35|length: 12 bp
gataattcttta
>34|random sequence|A: 0.35|C: 0.15|G: 0.15|T: 0.35|length: 19 bp
attagttgtttcaaatcta
>35|random sequence|A: 0.35|C: 0.15|G: 0.15|T: 0.35|length: 10 bp
tgcataattt
>36|random sequence|A: 0.35|C: 0.15|G: 0.15|T: 0.35|length: 10 bp
atgtttcaat
>37|random sequence|A: 0.35|C: 0.15|G: 0.15|T: 0.35|length: 10 bp
tagtttcata
>38|random sequence|A: 0.35|C: 0.15|G: 0.15|T: 0.35|length: 18 bp
tgattaaatgtacatctt
>39|random sequence|A: 0.35|C: 0.15|G: 0.15|T: 0.35|length: 19 bp
agactcttatttaagtatt
>40|random sequence|A: 0.35|C: 0.15|G: 0.15|T: 0.35|length: 18 bp
attgaagttcttcattaa
>41|random sequence|A: 0.35|C: 0.15|G: 0.15|T: 0.35|length: 8 bp
atttcatg
>42|random sequence|A: 0.35|C: 0.15|G: 0.15|T: 0.35|length: 17 bp
tccttatatatgattga
>43|random sequence|A: 0.35|C: 0.15|G: 0.15|T: 0.35|length: 18 bp
tataagttataactgctt
>44|random sequence|A: 0.35|C: 0.15|G: 0.15|T: 0.35|length: 19 bp
ctctattaatatatagttg
>45|random sequence|A: 0.35|C: 0.15|G: 0.15|T: 0.35|length: 8 bp
tcagttat
>46|random sequence|A: 0.35|C: 0.15|G: 0.15|T: 0.35|length: 12 bp
atttatttacag
>47|random sequence|A: 0.35|C: 0.15|G: 0.15|T: 0.35|length: 12 bp
atatattagtct
>48|random sequence|A: 0.35|C: 0.15|G: 0.15|T: 0.35|length: 19 bp
attctgaatatgttacatt
>49|random sequence|A: 0.35|C: 0.15|G: 0.15|T: 0.35|length: 10 bp
catattatgt
>50|random sequence|A: 0.35|C: 0.15|G: 0.15|T: 0.35|length: 17 bp
ataattagctttgtcta
>51|random sequence|A: 0.35|C: 0.15|G: 0.15|T: 0.35|length: 10 bp
tgttcttaaa
>52|random sequence|A: 0.35|C: 0.15|G: 0.15|T: 0.35|length: 17 bp
tgttttaacattgacta
>53|random sequence|A: 0.35|C: 0.15|G: 0.15|T: 0.35|length: 9 bp
ttgttaaca
>54|random sequence|A: 0.35|C: 0.15|G: 0.15|T: 0.35|length: 11 bp
tcttttatgaa
>55|random sequence|A: 0.35|C: 0.15|G: 0.15|T: 0.35|length: 14 bp
gtatcatttgctaa
>56|random sequence|A: 0.35|C: 0.15|G: 0.15|T: 0.35|length: 12 bp
tctattaattag
>57|random sequence|A: 0.35|C: 0.15|G: 0.15|T: 0.35|length: 11 bp
tatcttgttaa
>58|random sequence|A: 0.35|C: 0.15|G: 0.15|T: 0.35|length: 10 bp
ttatcgaatt
>59|random sequence|A: 0.35|C: 0.15|G: 0.15|T: 0.35|length: 12 bp
tgaactatttat
>60|random sequence|A: 0.35|C: 0.15|G: 0.15|T: 0.35|length: 9 bp
ctaaagttt
>61|random sequence|A: 0.35|C: 0.15|G: 0.15|T: 0.35|length: 18 bp
aatatagtcttcttgata
>62|random sequence|A: 0.35|C: 0.15|G: 0.15|T: 0.35|length: 19 bp
aaggcaattttattcattt
>63|random sequence|A: 0.35|C: 0.15|G: 0.15|T: 0.35|length: 9 bp
gttcaatta
>64|random sequence|A: 0.35|C: 0.15|G: 0.15|T: 0.35|length: 20 bp
aagtttcgatattagaatcc
>65|random sequence|A: 0.35|C: 0.15|G: 0.15|T: 0.35|length: 10 bp
tgtctataat
>66|random sequence|A: 0.35|C: 0.15|G: 0.15|T: 0.35|length: 11 bp
ttcaattttga
>67|random sequence|A: 0.35|C: 0.15|G: 0.15|T: 0.35|length: 13 bp
tttcattgaaatt
>68|random sequence|A: 0.35|C: 0.15|G: 0.15|T: 0.35|length: 11 bp
aatgtctttat
>69|random sequence|A: 0.35|C: 0.15|G: 0.15|T: 0.35|length: 20 bp
agaaattgaccattgtactt
>70|random sequence|A: 0.35|C: 0.15|G: 0.15|T: 0.35|length: 19 bp
ttaactcgttttattagaa
>71|random sequence|A: 0.35|C: 0.15|G: 0.15|T: 0.35|length: 20 bp
agatcatacgagcattattt
>72|random sequence|A: 0.35|C: 0.15|G: 0.15|T: 0.35|length: 13 bp
aattttctaatgt
>73|random sequence|A: 0.35|C: 0.15|G: 0.15|T: 0.35|length: 18 bp
ttatttgataccttgaaa
>74|random sequence|A: 0.35|C: 0.15|G: 0.15|T: 0.35|length: 8 bp
ctagttta
>75|random sequence|A: 0.35|C: 0.15|G: 0.15|T: 0.35|length: 14 bp
gactttcaagttta
>76|random sequence|A: 0.35|C: 0.15|G: 0.15|T: 0.35|length: 11 bp
tcatgatttat
>77|random sequence|A: 0.35|C: 0.15|G: 0.15|T: 0.35|length: 19 bp
tttaatttgttagaactca
>78|random sequence|A: 0.35|C: 0.15|G: 0.15|T: 0.35|length: 20 bp
ctattttaggaaacacattg
>79|random sequence|A: 0.35|C: 0.15|G: 0.15|T: 0.35|length: 14 bp
cgattatctgaatt
>80|random sequence|A: 0.35|C: 0.15|G: 0.15|T: 0.35|length: 16 bp
aaaatcttagtcgttt
>81|random sequence|A: 0.35|C: 0.15|G: 0.15|T: 0.35|length: 10 bp
ttacgattat
>82|random sequence|A: 0.35|C: 0.15|G: 0.15|T: 0.35|length: 9 bp
ctatgatat
>83|random sequence|A: 0.35|C: 0.15|G: 0.15|T: 0.35|length: 12 bp
aattattctagt
>84|random sequence|A: 0.35|C: 0.15|G: 0.15|T: 0.35|length: 9 bp
tttagtaac
>85|random sequence|A: 0.35|C: 0.15|G: 0.15|T: 0.35|length: 17 bp
gcgttttaatatacatt
>86|random sequence|A: 0.35|C: 0.15|G: 0.15|T: 0.35|length: 9 bp
aaagttttc
>87|random sequence|A: 0.35|C: 0.15|G: 0.15|T: 0.35|length: 14 bp
gtaacttcgttaat
>88|random sequence|A: 0.35|C: 0.15|G: 0.15|T: 0.35|length: 13 bp
cttattaatagtt
>89|random sequence|A: 0.35|C: 0.15|G: 0.15|T: 0.35|length: 14 bp
gatttacatttgac
>90|random sequence|A: 0.35|C: 0.15|G: 0.15|T: 0.35|length: 12 bp
ttttaacattag
>91|random sequence|A: 0.35|C: 0.15|G: 0.15|T: 0.35|length: 12 bp
ctaatttgatta
>92|random sequence|A: 0.35|C: 0.15|G: 0.15|T: 0.35|length: 10 bp
tgtctaatta
>93|random sequence|A: 0.35|C: 0.15|G: 0.15|T: 0.35|length: 16 bp
ttactttaaagttacg
>94|random sequence|A: 0.35|C: 0.15|G: 0.15|T: 0.35|length: 12 bp
tatatgctttaa
>95|random sequence|A: 0.35|C: 0.15|G: 0.15|T: 0.35|length: 19 bp
ttcttgatacatataagtt
>96|random sequence|A: 0.35|C: 0.15|G: 0.15|T: 0.35|length: 13 bp
tatattcgatatt
>97|random sequence|A: 0.35|C: 0.15|G: 0.15|T: 0.35|length: 16 bp
gtctcatgttttaaaa
>98|random sequence|A: 0.35|C: 0.15|G: 0.15|T: 0.35|length: 17 bp
agttttaactgatcatt
>99|random sequence|A: 0.35|C: 0.15|G: 0.15|T: 0.35|length: 9 bp
tgtatcata
>100|random sequence|A: 0.35|C: 0.15|G: 0.15|T: 0.35|length: 10 bp
agttaatctt

As will be apparent to one of ordinary skill in the art from a reading of this disclosure, the present disclosure can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

What is claimed is:

1. A method of detecting nucleic acid fragments, the method comprising:
providing a plurality of chambers, each chamber separated from the other chambers of the plurality, each chamber having at least one set of probes disposed therein, each set of probes being capable of binding to a different target nucleic acid sequence relative to the other sets of probes;
providing a sample comprising a plurality of sets of nucleic acid fragments;
placing at least a portion of the sample into each of the plurality of chambers and causing the at least one set of probes in each chamber to bind with complementary nucleic acid fragments of the sample; and
detecting the binding of the nucleic acid fragments to the sets of probes in each chamber.

2. The method of claim 1, wherein the placing the at least a portion of the sample into each of the plurality of chambers comprises flowing the portions of the sample through a first chamber of the plurality of chambers, the chambers being fluidically coupled in series.

3. The method of claim 1, wherein the placing the at least a portion of the sample into each of the plurality of chambers comprises flowing the portions of the sample into the plurality of chambers, the chambers being fluidically coupled in parallel.

4. The method of claim 1, wherein the nucleic acid fragments are DNA fragments.

5. The method of claim 1, wherein the nucleic acid fragments are RNA fragments.

6. The method of claim 1, wherein the plurality of chambers are disposed in a biochip, the biochip comprising an input port in fluid communication with the plurality of chambers.

7. The method of claim 1, wherein providing the sample comprises performing a nucleic acid amplification.

8. The method of claim 7, wherein the nucleic acid amplification comprises at least one of PCR amplification and isothermal amplification.

9. The method of claim 1, wherein the sets of probes are fluorescently labeled, the binding of the complementary nucleic acid fragments of the sample persevering the fluorescence of the label, and wherein the detecting the binding of the nucleic acid fragments to the sets of probes includes detecting the fluorescence of the labels.

10. The method of claim 9, further comprising quenching the fluorescent labels of unbound probes.

11. The method of claim 9, wherein the sets of probes are immobilized within the chambers.

12. The method of claim 1, wherein providing the sample comprises performing a nucleic acid amplification in which copies of amplified nucleic acid fragments are fluorescently labeled, the binding of the complementary nucleic acid fragments of the sample persevering the fluorescence of the nucleic acid fragments, and wherein the detecting the binding of the nucleic acid fragments to the sets of probes includes detecting the fluorescence of the nucleic acid fragments.

13. The method of claim 12, further comprising quenching the fluorescent labels of unbound nucleic acid fragments.

14. The method of claim 12, further comprising washing unbound nucleic acid fragments out of the plurality of plurality of chambers.

15. The method of claim 12, wherein the sets of probes are immobilized within the chambers.

16. The method of claim 1, wherein:
providing the sample comprises performing a nucleic acid amplification in which copies of amplified nucleic acid fragments are amplified using fluorescently labeled bases of a specified type;
at least one of the sets of probes has fluorescently labeled bases that are complementary to the specified type, the fluorescently labeled bases of the probes being quenched by binding to the fluorescently labeled bases of the nucleic acid fragments; and
the detecting the binding of the nucleic acid fragments to the sets of probes includes detecting when a fluorescently labeled base of the probes is not quenched.
